(19) United States
(12) Patent Application Publication
(10) Pub. No.: US 2025/0352557 A1
(43) Pub. Date: Nov. 20, 2025

(54) COMPOSITIONS FOR PERITONEAL DRUG DELIVERY AND METHODS OF USE THEREOF

(71) Applicant: Viramal Limited, London (GB)

(72) Inventors: William BOLOGNA, New York, NY (US); Finn LARSEN, London (GB); Simona FIORE, London (GB)

(21) Appl. No.: 19/283,695

(22) Filed: Jul. 29, 2025

Related U.S. Application Data
(63) Continuation of application No. PCT/US2024/013600, filed on Jan. 30, 2024.
(60) Provisional application No. 63/442,054, filed on Jan. 30, 2023.

(51) Int. Cl. A61K 31/58; A61K 9/00; A61K 9/06; A61K 9/107; A61K 47/32; A61K 47/38; A61P 15/08

(52) U.S. Cl. CPC A61K 31/58; A61K 9/0034; A61K 9/06; A61K 9/107; A61K 47/32; A61K 47/38; A61P 15/08

(57) ABSTRACT

Disclosed herein are compositions for administering vaginally of an active agent or a salt to a subject comprising the active agent or salt thereof, a bioadhesive, and an emulsion. Provided herein are methods treating a disease of condition by administering vaginally to a subject a pharmaceutical composition, and kits comprising the pharmaceutical composition. Further provided are methods of localizing an active agent to the peritoneal cavity of a subject while minimizing systemic circulation of the active agent.

### CROSS-REFERENCE TO RELATED APPLICATIONS

This application is a continuation application of PCT Application No. PCT/US2024/013600, filed Jan. 30, 2024, which claims the benefit of priority to U.S. Provisional Patent Application 63/442,054 filed Jan. 30, 2023, the contents of which are incorporated herein by reference in their entirety.

### BACKGROUND

Endometriosis is a condition in women characterized by the presence of endometrial tissue (endometrial glands and stroma) outside the uterine cavity and can commonly be associated with chronic pelvic pain and infertility. An example of these lesions can be peritoneal lesions or deep infiltrating disease. Nearly 50% of infertile women are affected by endometriosis. Unfortunately, the efficacy of treatments for endometriosis and other endometrial disorders includes hormone therapy and analgesics that treat symptomatic endometriosis is limited and endometriosis has a high rate of recurrence in many women. Therefore, there is a great unmet need for therapeutics that alleviate and prevent endometrial diseases and related disorders long-term while reducing systemic side effects associated with the oral administration of current therapeutics.

### BRIEF SUMMARY

Provided herein are compositions, methods, and kits for delivering an agent to the peritoneal cavity.

Provided herein are compositions comprising a cream, a polybioadhesive gel, or a liquid emulsion provided herein.

Provided herein are creams, wherein the creams comprise: (a) micronized danazol or a salt thereof that is present in an amount of 5.0% up to about 7.0% weight to volume (w/v) of the cream; (b) polyglycolyzed oleic glyceride that is present in an amount of 10% up to 20% (w/v) of the cream; (c) a cellulose polymer that is present in an amount of 1.0% up to 3.0% (w/v) of the cream; and (d) one or more water-insoluble bioadhesives, wherein at least one water-insoluble bioadhesive is polycarbophil that is present in an amount of 1.0% up to about 2.0% (w/v) of the cream, wherein the cream comprises at least 60% (w/v) water and comprises one or more ingredients selected from: a carbomer, methylparaben, sorbic acid, a poloxamer, and a sodium carboxymethylcellulose.

Provided herein are compositions, wherein the compositions comprise: a polybioadhesive gel comprising: (a) an active agent or a salt thereof; (b) one or more water-insoluble bioadhesive; (c) at least one oleogel; and (d) at least one aqueous gel, wherein upon administration to a subject, the active agent localizes to the peritoneal tissue and induces pelvic pass metabolism in the subject.

Provided herein are compositions comprising the components of Table 3 and a respective quantity (w/v) of each component.

Provided herein are methods for treating an endometrial disease or condition in a subject, wherein the methods comprise: vaginally administering to the subject a polybioadhesive gel comprising: (a) an active agent or a salt thereof; (b) one or more water-insoluble bioadhesives; (c) at least one oleogel; and (d) at least one aqueous gel, wherein vaginally administering the polybioadhesive gel localizes the active agent to the peritoneal tissue, thereby treating the endometrial disease or condition.

Provided herein are methods of inducing pelvic pass metabolism of an active agent in a subject, wherein the methods comprise: vaginally administering to a subject a polybioadhesive gel comprising: (a) an active agent or a salt thereof; (b) one or more water-insoluble bioadhesive; (c) at least one oleogel; and (d) at least one aqueous gel, wherein the vaginally administering increases the level of the active agent in the peritoneal tissue relative to the level of the active agent in the serum of the subject, and wherein the vaginally administering maintains a ratio of the level of the active agent in a serum of the subject to the level of the active agent in a peritoneal fluid of the subject that is substantially the same relative to oral administration of an oral pharmaceutical composition comprising a substantially equivalent dose of the active agent or salt thereof, thereby localizing the active agent to the peritoneal tissue and inducing pelvic pass metabolism in the subject.

Provided herein are methods of treating a disease or a condition in a subject, wherein the methods comprise: vaginally administering to the subject a cream provided herein or a composition provided herein, thereby treating the disease or the condition.

Provided herein are methods of maintaining ovarian function and reducing pain associated with endometriosis in a subject, wherein the methods comprise: vaginally administering to the subject a cream provided herein or a composition provided herein, thereby maintaining ovarian function and reducing pain associated with endometriosis in a subject.

Provided herein are methods for treating an endometrial disease or condition in a subject, wherein the methods comprise: vaginally administering to the subject a polybioadhesive gel comprising: (a) an active agent or a salt thereof; (b) one or more water-insoluble bioadhesives; (c) at least one oleogel; and (d) at least one aqueous gel, wherein: the vaginally administering the polybioadhesive gel reduces systemic concentration of the active agent or the salt thereof in the subject by at least 30-fold relative to oral administration of the active agent or the salt thereof to a subject; and the vaginally administering the polybioadhesive gel localizes the active agent to a peritoneal tissue of the subject, thereby treating the endometrial disease or condition in the subject.

Provided herein are methods of maintaining ovarian function and reducing pain associated with endometriosis in a subject, wherein the methods comprise: vaginally administering to the subject a polybioadhesive gel comprising: (a) an active agent or a salt thereof; (b) one or more water-insoluble bioadhesives; (c) at least one oleogel; and (d) at least one aqueous gel, thereby maintaining ovarian function and reducing pain associated with endometriosis in the subject, wherein: the vaginally administering the polybioadhesive gel reduces systemic concentration of the active agent or the salt thereof in the subject by at least 30-fold relative to oral administration of the active agent or the salt thereof to a subject; and the vaginally administering the polybioadhesive gel localizes the active agent to a peritoneal tissue of the subject.

### BRIEF DESCRIPTION OF THE DRAWINGS

The novel features are set forth with particularity in the appended claims. A better understanding of features and advantages will be obtained by reference to the following detailed description that sets forth illustrative embodiments, in which exemplary principles are utilized, and the accompanying drawings of which:

FIG. 1 depicts an illustration of the peritoneal cavity and organs contained therein.

FIG. 2 illustrates the VML-0501-001 study design and timeline.

FIG. 3 shows a graph of the ratio of Danazol concentration between Serum and Peritoneal fluid in the (ITT) population. X-axis: Conditions; Y-axis: Plasma ratio.

FIG. 4 shows a graph of the ratio of Danazol concentration between Serum and Peritoneal fluid in the PP population. X-axis: Conditions; Y-axis: Plasma ratio.

FIG. 5 shows a graph of Danazol in Serum and tissue concentration in VML-0501 dosed group. X-axis: Conditions; Y-Axis: Concentration.

FIG. 6 shows a graph of the concentration of danazol in Serum and Tissue in Oral Danazol group. X-axis: Conditions; Y-Axis: Concentration.

FIG. 7 shows a graph of the concentration of danazol in the peritoneal tissue in the vaginally administered group. X-axis: Conditions; Y-Axis: Concentration. N=6.

### DETAILED DESCRIPTION

Disclosed herein are compositions and methods for delivering drug vaginally, in order to reach the pelvic area in such a way that high peritoneal fluid and tissue concentrations can be achieved with low detectable levels in the systemic circulation. In some cases, concentrations of at least 2 ng/ml, at least 5 ng/ml; at least >10 ng/ml, or even greater can be achieved. Comparing to other delivery method (e.g., oral administration), the drug delivery method disclosed herein can improve therapeutic efficacy. In some cases, such a delivery can maintain a substantially zero-order release profile of a therapeutically effective amount of an active agent or salt thereof into a peritoneal fluid for a given time interval when applied vaginally to a subject.

Provided herein are methods of achieving a desired pharmacokinetic profile upon vaginally delivering to a subject a pharmaceutical composition. In some cases, the pharmaceutical composition can comprise an emulsion that can be admixed with an active agent and a bioadhesive. In some embodiments, the bioadhesive is water-insoluble. In some embodiments, the water-insoluble bioadhesive is dispersed in at least a portion of the aqueous phase.

An administering vaginally of a pharmaceutical composition described herein can be used to locally deliver an active agent or a salt thereof to a peritoneal cavity. Such a deliver can be used in lieu of systemic delivery, thereby decreasing an amount of the active agent or salt thereof present in circulation after administering vaginally of the pharmaceutical composition, relative to a systemic administering (such as orally administering) of a pharmaceutical composition that can comprise a substantially equivalent dose of the active agent or salt thereof. In some embodiments, the method described herein can reduce a potential for systemic adverse events and unwanted side effects that can occur with systemic administering of an active agent or salt thereof. Detection of an amount of an active agent or salt thereof in a subject sample is also contemplated using an assay employing detection of an albumin conjugate using mass spectrometry.

Administering vaginally of a pharmaceutical composition can be used to treat a disease or a condition in a subject. In some cases, a subject can be a subject in need of such a treatment, such as a subject who may be suspected of harboring, or has previously been diagnosed with, a disease or condition treatable by administering vaginally to the subject a pharmaceutical composition described herein. A pharmaceutical composition described herein can include at least one active agent that can be selected based on the disease or condition to be treated.

Provided herein are kits that can comprise a pharmaceutical composition described herein. Such a kit can include instructions for application of a pharmaceutical composition, and means for applying the pharmaceutical composition.

### Definitions

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

As used herein, the term “about” or “approximately” means a range of up to +20% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “effective amount” or “therapeutically effective amount” refers to an amount that is sufficient to achieve or at least partially achieve the desired effect.

The term “zero order release rate profile” or “zero order release profile” can refer to a profile in which a concentration of an active agent can be released into a vasculature of a subject at a constant rate over a given time interval. In some cases, an active agent can have a substantially zero order release profile into a serum, lymph, or peritoneal vasculature of a subject upon administering of the pharmaceutical composition.

As used herein, the term “bioavailability” can denote the degree to which a drug such as an active agent, salt, metabolite, or other substance becomes available to the target tissue after administration.

Parameters often used in pharmacokinetic (PK) studies can include Tmax, Cmax, AUC(0-∞), AUC(0-t), and T_{1/2} and CL/F. “Tmax” can refer to the time to reach the maximal plasma concentration (“Cmax”) after administration of a therapeutic; “AUC(0-∞)” can refer to the area under the plasma concentration versus time curve from time 0 to infinity; “AUC(0-t)” can refer to the area under the plasma concentration versus time curve from time 0 to time t; “T_{1/2}” can refer to a half-life of a therapeutic in blood plasma; “T_{1/2, elim}” can refer to the half-life of elimination of the therapeutic from circulation; and “CL/F” can refer to an apparent clearance rate of a therapeutic.

### Compositions

Provided herein are compositions for localized delivery of an active agent or a salt thereof. In some embodiments, a composition comprises an emulsion which can be a substantially uniform mixture of an organic phase and an aqueous phase, an active agent or a salt thereof, and a bioadhesive.

In some embodiments, a composition provided herein can be formulated for vaginal delivery. Formulation of a pharmaceutical composition can include admixing an organic phase and aqueous phase with a bioadhesive and an active agent or salt thereof. In order to improve the delivery an active agent or a salt thereof, bioadhesives can be used to adhere a pharmaceutical composition to an epithelial surface upon administering vaginally of the pharmaceutical composition.

The inventors discovered the surprising and unexpected result that a pharmaceutical composition as described herein comprising a liquid emulsion can prevent or minimize vaginal clumping or discharge when formulated with a bioadhesive by emulsifying the insoluble material, thereby minimizing or eliminating unsightly clumping or discharge. Such an emulsion can comprise a substantially uniform mixture of an organic phase and an aqueous phase containing an active agent or salt thereof and a bioadhesive. It is envisaged that an aqueous phase and an organic phase can be provided as a uniform mixture, or that each component can be provided separately for mixture prior to administration.

An emulsion containing an organic phase and an aqueous phase can contain various components or ingredients. In some cases, an organic phase can contain at least one oleogel. An oleogel can comprise at least one oily agent and at least one polymer.

An oily agent can be a monoglyceride, a diglyceride, a triglyceride, or any combination thereof. In some cases, an oily agent can be isolated and purified. In some cases, an oily agent can be a synthetic diglyceride, a synthetic triglyceride, a propylene glycol isostearate, a polyoxyethylenated oleic glyceride mixture, an oil of plant or natural origin, or any combination thereof.

In some cases, an oily agent can be present in a proportion of from about 0.1 to about 1%, 0.1 to about 2%, 0.1 to about 3%, 0.1 to about 4%, 0.1 to about 5%, 0.1 to about 6%, 0.1 to about 7%, 0.1 to about 8%, 0.1 to about 9%, 0.1 to about 10%, 0.1 to about 11%, 0.1 to about 12%, 0.1 to about 13%, 0.1 to about 14%, 0.1 to about 15%, 0.1 to about 16%, 0.1 to about 17%, 0.1 to about 18%, 0.1 to about 19%, 0.1 to about 20%, 0.1 to about 21%, 0.1 to about 22%, 0.1 to about 23%, 0.1 to about 24%, 0.1 to about 25%, 0.1 to about 26%, 0.1 to about 27%, 0.1 to about 28%, 0.1 to about 29%, or 0.1 to about 30% of the weight, relative to the weight of the organic phase.

In some cases, an oily agent can be present in a proportion of from about 0.01 to about 0.1%, 0.01 to about 0.2%, 0.01 to about 0.3%, 0.01 to about 0.4%, 0.01 to about 0.5%, 0.01 to about 0.6%, 0.01 to about 0.7%, 0.01 to about 0.8%, 0.01 to about 0.9%, 0.01 to about 1%, 0.01 to about 2%, 0.01 to about 3%, 0.01 to about 4%, 0.01 to about 5%, 0.01 to about 6%, 0.01 to about 7%, 0.01 to about 8%, 0.01 to about 9%, 0.01 to about 10%, 0.01 to about 11%, 0.01 to about 12%, 0.01 to about 13%, 0.01 to about 14%, 0.01 to about 15%, 0.01 to about 16%, 0.01 to about 17%, 0.01 to about 18%, 0.01 to about 19%, 0.01 to about 20%, 0.01 to about 21%, 0.01 to about 22%, 0.01 to about 23%, 0.01 to about 24%, 0.01 to about 25%, 0.01 to about 26%, 0.01 to about 27%, 0.01 to about 28%, 0.01 to about 29%, or 0.01 to about 30% of the weight, relative to the weight of the composition.

In some exemplary embodiments, an oily agent can be propylene glycol isostearate. Exemplary pharmaceutical compositions can comprise propylene glycol isostearate in a proportion of between of about 5 and 90% by weight, relative to the total weight of the oleogel.

A mono-di- or triglyceride can be a molecule of Formula I:

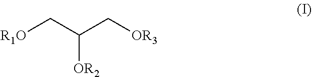

where R₁, R₂, and R₃ can independently be H; or C₁-C₂₀ alkyl comprising 0, 1, 2, 3, 4 or 5 degrees of unsaturation.

In some aspects, the synthetic mono-di- or triglyceride can be “LABRAFAC® lipophile WL1349”, sold by the company Gatefosse, propylene glycol isostearate, such as the product sold under the name “hydrophilol isostearique” by the company Gatefosse, and the polyglycolyzed glyceride “LABRRAFIL® M 1944 CS” as sold by Gatefosse.

LABRRAFIL® M 1944 CS is a mixture of polyoxyethylenated oleic glycerides obtained by the alcoholysis of natural plant oil. It can be an oily liquid whose properties are presented in Table 1 below.

TABLE 1
Polyoxyethylenated oleic glyceride properties.

| Chemical Name | Polyglycolyzed oleic glyceride |
|---|---|
| Trade name | LABRRAFIL ® M 1944 CS |
| Drop Point ° C. | Liq. |
| Saponification Number | 1451175 |
| Acid number | >2 |
| Iodine Number | 60/90 |
| Oral acute toxicity rat | OLD > 20 mg/Kg |
| LOP | 0 |
| HLB | 314 |

In some cases, a mono-, di- or triglyceride can be of natural or plant origin. An oil of natural or plant origin can include an oil such as sweet almond oil, argan oil or palm oil.

A polymer in an organic phase can be a cellulose polymer. In some cases, a cellulose polymer can bean ethylcellulose, a non-sodium containing carboxymethylcellulose or a mixture thereof. In some cases, the polymer can be a water insoluble polymer. In some exemplary embodiments, a water insoluble polymer can be a water insoluble cellulose polymer.

A cellulose polymer can be a lipid soluble cellulose polymer. In some instance, the cellulose polymer can be an alkyl cellulose. In some instance, the alkyl cellulose can be methylcellulose, ethylcellulose, hydroxypropylcellulose or a combination thereof. In some embodiments, the cellulose polymer can be an alkyl carboxylic containing cellulose or a salt thereof. In some cases, the alkyl carboxylic containing cellulose can be non-sodium containing carboxymethylcellulose.

In some cases, the water insoluble polymer can be present in a proportion of from about 0.1 to about 1%, 0.1 to about 2%, 0.1 to about 3%, 0.1 to about 4%, 0.1 to about 5%, 0.1 to about 6%, 0.1 to about 7%, 0.1 to about 8%, 0.1 to about 9%, 0.1 to about 10%, 0.1 to about 11%, 0.1 to about 12%, 0.1 to about 13%, 0.1 to about 14%, 0.1 to about 15%, 0.1 to about 16%, 0.1 to about 17%, 0.1 to about 18%, 0.1 to about 19%, 0.1 to about 20%, 0.1 to about 21%, 0.1 to about 22%, 0.1 to about 23%, 0.1 to about 24%, 0.1 to about 25%, 0.1 to about 26%, 0.1 to about 27%, 0.1 to about 28%, 0.1 to about 29%, or 0.1 to about 30% of the weight, relative to the weight of the organic phase.

In some cases, the water insoluble polymer can be present in a proportion of from about 0.01 to about 0.1%, 0.01 to about 0.2%, 0.01 to about 0.3%, 0.01 to about 0.4%, 0.01 to about 0.5%, 0.01 to about 0.6%, 0.01 to about 0.7%, 0.01 to about 0.8%, 0.01 to about 0.9%, 0.01 to about 1%, 0.01 to about 2%, 0.01 to about 3%, 0.01 to about 4%, 0.01 to about 5%, 0.01 to about 6%, 0.01 to about 7%, 0.01 to about 8%, 0.01 to about 9%, 0.01 to about 10%, 0.01 to about 11%, 0.01 to about 12%, 0.01 to about 13%, 0.01 to about 14%, 0.01 to about 15%, 0.01 to about 16%, 0.01 to about 17%, 0.01 to about 18%, 0.01 to about 19%, 0.01 to about 20%, 0.01 to about 21%, 0.01 to about 22%, 0.01 to about 23%, 0.01 to about 24%, 0.01 to about 25%, 0.01 to about 26%, 0.01 to about 27%, 0.01 to about 28%, 0.01 to about 29%, or 0.01 to about 30% of the weight, relative to the weight of the composition.

In some exemplary embodiments, a cellulose polymer can be present in a proportion of 1 and about 10% by weight. In some exemplary embodiments, a cellulose polymer can be ethylcellulose present in a proportion of 1 and about 10% by weight based on a total weight of a composition. In some cases, an oily agent can comprise LABRRAFIL® M 1944 CS. In some cases, the oily agent can be present in a proportion of between about 5 and 90% by weight, relative to the total weight of the oleogel. In some cases, the ratio of the oleogel to the weight of the aqueous gel can be from about 10:90 to about 90:10. In some cases, the cellulose polymer can be EMULFREE® P. In some embodiments, the cellulose polymer can be EMULFREE® P and the oily agent can comprise LABRRAFIL® M 1944 CS.

An aqueous phase as described herein can comprise one or more aqueous gels. The aqueous phase can comprise at least one aqueous gel. In some cases, an aqueous gel can comprise at least one gelling agent. A gelling agent may be a carbomer, a poloxamer, a sodium carboxymethylcellulose, or any mixtures thereof.

In some embodiments, the gelling agent can be present in a proportion of from about 0.1 to about 1%, 0.1 to about 2%, 0.1 to about 3%, 0.1 to about 4%, 0.1 to about 5%, 0.1 to about 6%, 0.1 to about 7%, 0.1 to about 8%, 0.1 to about 9%, 0.1 to about 10%, 0.1 to about 11%, 0.1 to about 12%, 0.1 to about 13%, 0.1 to about 14%, 0.1 to about 15%, 0.1 to about 16%, 0.1 to about 17%, 0.1 to about 18%, 0.1 to about 19%, 0.1 to about 20%, 0.1 to about 21%, 0.1 to about 22%, 0.1 to about 23%, 0.1 to about 24%, 0.1 to about 25%, 0.1 to about 26%, 0.1 to about 27%, 0.1 to about 28%, 0.1 to about 29%, or 0.1 to about 30% of the weight, relative to the weight of the aqueous phase.

In some cases, a gelling agent can be present in a proportion of from about 0.01 to about 0.1%, 0.01 to about 0.2%, 0.01 to about 0.3%, 0.01 to about 0.4%, 0.01 to about 0.5%, 0.01 to about 0.6%, 0.01 to about 0.7%, 0.01 to about 0.8%, 0.01 to about 0.9%, 0.01 to about 1%, 0.01 to about 2%, 0.01 to about 3%, 0.01 to about 4%, 0.01 to about 5%, 0.01 to about 6%, 0.01 to about 7%, 0.01 to about 8%, 0.01 to about 9%, 0.01 to about 10%, 0.01 to about 11%, 0.01 to about 12%, 0.01 to about 13%, 0.01 to about 14%, 0.01 to about 15%, 0.01 to about 16%, 0.01 to about 17%, 0.01 to about 18%, 0.01 to about 19%, 0.01 to about 20%, 0.01 to about 21%, 0.01 to about 22%, 0.01 to about 23%, 0.01 to about 24%, 0.01 to about 25%, 0.01 to about 26%, 0.01 to about 27%, 0.01 to about 28%, 0.01 to about 29%, or 0.01 to about 30% of the weight, relative to the weight of the composition.

In some aspects, a gelling agent for the aqueous phase can be a carbomer, Carbopol 974 or Carbopol 980, present in a proportion of between of about 0.1 and about 5% by weight, relative to the total weight of the aqueous phase.

In some cases, the ratio of the weight of the organic phase to the weight of the aqueous phase can be from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, from about 1:2 to about 2:1, or from about 1:1.5 to about 1.5:1. In some cases, the ratio of the weight of the organic phase to the weight of the aqueous phase can be about 1:1. In some aspects, an emulsion in the composition herein can comprise a substantially uniform mixture of an organic phase and an aqueous phase. In some cases, the ratio of the weight of the organic phase to the weight of the aqueous phase in such a substantially uniform mixture can be about 1:1.

In some cases, a uniform mixture of an organic phase and an aqueous phase can maintain a stable uniform appearance over a period of time when stored in a sealed container. In some cases, the mixture can be stable for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks. In some cases, the mixture can be stable for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, months. In some cases, the mixture can be stable for at least about 1, 2, 3, 4, 5, 6, 7, 8, 9, or 10 years. In some cases, the sealed container can be stored at a temperature of about 25° C. at about 1 atm pressure. In some cases, the sealed container can be stored at about 25, 30, 35, 40, 45, 50, 55, 60, 65, 70, 75, 80, 85, 90, 95, 96, 97, 98, 99, or 100% relative humidity.

In some cases, the ratio of the volume of the organic phase to the volume of the aqueous phase can be from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, from about 1:2 to about 2:1, or from about 1:1.5 to about 1.5:1. In some cases, the ratio of the volume of the organic phase to the volume of the aqueous phase can be about 1:1.

An emulsion can include dispersions or droplets, as well as other lipid structures that can form as a result of hydrophobic forces that drive a polar residue (e.g., long hydrocarbon chains) away from water and drive polar head groups toward water, when a water immiscible oily phase can be mixed with an aqueous phase. These other lipid structures can include unilamellar, paucilamellar, multilamellar lipid vesicles, micelles, and lamellar phases.

In some cases, a penetration enhancer can be present in an amount sufficient to achieve an enhanced rate of penetration. In some cases, a penetration enhancer does not enhance a rate of penetration of an active agent or salt thereof below a threshold concentration. A threshold concentration can be specific to a given penetration enhancer. In some cases, a penetration enhancer enhances a rate of absorption of an active agent or salt thereof when present at a concentration by weight of at least about 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% by weight relative to a total weight of a composition.

In some cases, a composition described herein does not contain a penetration enhancer. In some cases, a composition described herein contains no more than about 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% by weight of a penetration enhancer relative to a total weight of a composition.

The oleogel and the aqueous phase can respectively further comprise standard ingredients for a gel, such as texture agents, antioxidants, preservatives, dyes or fragrances of various types and in conventional amounts which are known to not cause skin irritation.

The composition can be in the form of a pharmaceutical composition. In some embodiments, the pharmaceutical composition can be administered similar to a cosmetic product in the form of a cream or gel that can be applied to the skin. In some embodiments, the composition can be in a unit dose form when applied to at least a portion of a skin in a specified amount. Since the pharmaceutical composition can comprise a stable mixture of an oleo-gel and an aqueous gel, it can be neither messy nor watery, and in comparison to conventional hydroalcoholic gels, it requires a smaller area for application, and can be quicker to dry.

In some embodiments, the gel composition can comprise a bioadhesive. Examples of bioadhesives can include carbomers, glyceryl monooleate, hypromellose, polycarbophil, poly(methylvinyl ether-co-maleic anhydride), as well as salts thereof. In some cases, a pharmaceutical composition can contain one or more bioadhesives. In some cases, a pharmaceutical composition can contain at least 1, 2, 3, 4, 5, 6, 7, 8, 9, or 10 bioadhesives.

In some exemplary embodiments, a bioadhesive can be polycarbophil or a salt thereof. Polycarbophil was designed to mimic negatively charged mucin, the glycoprotein component of mucus that is responsible for its attachment to underlying epithelial surfaces. Polycarbophil is a lightly cross-linked polymer. Polycarbophil is also a weak polyacid containing multiple carboxyl radicals (COO—) the source of its negative charges. These acid radicals can permit hydrogen bonding with a cell surface. Hydrogen bonds can be weak, in the case of polycarbophil they can be numerous. Bioadhesives such as polycarbophil can stay attached to vaginal epithelial cells until they turn over, which can be up to 7 days in menopausal women. However, vaginal clumping and discharge can occur due to the water insoluble polycarbophil remaining attached to the vaginal epithelial cells. Therefore, there is a need to provide a formulation that can provide an adherence of a pharmaceutical composition to an epithelium of a subject while minimizing vaginal clumping or discharge.

In some cases, a bioadhesive can be present at a concentration by weight of at least about 0.01%, at least about 0.05%, at least about 0.1%, at least about 0.15%, at least about 0.2%, at least about 0.25%, at least about 0.3%, at least about 0.35%, at least about 0.4%, at least about 0.45%, at least about 0.5%, at least about 0.55%, at least about 0.6%, at least about 0.65%, at least about 0.7%, at least about 0.75%, at least about 0.8%, at least about 0.85%, at least about 0.9%, at least about 0.95%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, or at least about 10%. In some embodiments, an aqueous gel can comprise a polycarbophil at a concentration by weight of from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.1% to about 0.9%, from about 0.1% to about 0.8%, from about 0.1% to about 0.7%, from about 0.1% to about 0.6%, from about 0.1% to about 0.5%, from about 0.1% to about 0.4%, from about 0.1% to about 0.3%, or from about 0.1% to about 0.2%.

The composition can comprise alcohol. For example, the alcohol can be a C₁-C₈ alcohol. Examples of a C₁-C₈ alcohol can include methanol, ethanol, n-propanol, isopropanol, t-butanol, pentanol, hexanol, cyclohexanol, heptanol, octanol and the like.

### Active Agents

A pharmaceutical composition as described herein can comprise at least one active agent or salt thereof. While exemplary active agents are described herein, it is possible substitute additional active agents in the composition in order to treat other indications treatable by administering of the pharmaceutical composition to a subject.

In some aspects, the active ingredients can be a hormone, an anti-inflammatory, an analgesic, a phenethylamine, an antineoplastic, a steroid, a 5-alpha reductase inhibitor, a gonadotropin-releasing hormone (GnRH) agonist, a GnRH antagonist, a glycine receptor antagonist, a tetrahydrocannabinol, an analgesic; an antibiotic, an antiviral compound, an antifungal compound, a salt of any of these, or any combination thereof.

In some embodiments, a hormone can be testosterone; dihydrotestosterone (DHT); estradiol; ethinylestradiol; progesterone; levonorgestrel; desogestrel; a peptide hormone such as oxytocin; a synthetic progesterone; a salt of any of these, or any combination thereof.

In some embodiments, an active ingredient can be an analgesic. Analgesics can include acetaminophen, bromfenac, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, nepafenac, oxaprozin, phenylbutazone, piroxicam, sulindac, tolmetin, celecoxib, buprenorphine, butorphanol, codeine, hydrocodone, hydromorphone, levorphanol, meperidine, methadone. Morphine, nalbuphine, oxycodone, oxymorphone, pentazocine, propoxyphene, tapentadol, tramadol, aspirin, a salt of any of these, or any combination thereof.

In some embodiments, a phenethylamine can be dopamine, epinephrine, norepinephrine, phenylephrine, methylphenidate, amphetamine, a salt of any of these, or any combination thereof.

In some embodiments, the antineoplastic can be cyclophosphamide, methotrexate, 5-fluorouracil, doxorubicin, procarbazine, prednisolone, bleomycin, vinblastine, dacarbazine, cisplatin, epirubicin, dichloroacetate, a salt of any of these, or any combination thereof.

In some embodiments, a steroid can be danazol or a salt thereof. Danazol is a synthetic androgen; a derivative of the synthetic steroid ethisterone. Chemically, Danazol, USP is 17α-Pregna-2,4-dien-20-yno [2,3-d]-isoxazol-17-ol.

In some embodiments, a 5-alpha reductase inhibitor can be dutasteride, tamsulosin, finasteride a salt of any of these, or any combination thereof.

In some embodiments, a GnRH agonist, a GnRH antagonist, can be leuprolide, buserelin, histrelin, goserelin, deslorelin, nafarelin, triptorelin, cetrorelix, abarelix; ganirelix, ozarelix, degarelix or teverelix a salt of any of these, or any combination thereof.

In some embodiments, a GnRH antagonist can be cetrorelix, abarelix; ganirelix, ozarelix, degarelix, teverelix, a salt of any of these, or any combination thereof.

In some embodiments, a glycine receptor antagonist can be tranexamic acid or a salt thereof.

In some embodiments, an antibiotic can be Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; or any combination thereof.

In some embodiments, an antiviral compound can be Ceftobiprole; Ceftaroline; Clindamycin; Dalbavancin; Daptomycin; Linezolid; Mupirocin; Oritavancin; Tedizolid; Telavancin; Tigecycline; Vancomycin; an Aminoglycoside; a Carbapenem; Ceftazidime; Cefepime; Ceftobiprole; a Fluoroquinolone; Piperacillin; Ticarcillin; Linezolid; a Streptogramin; Tigecycline; Daptomycin; a salt of any of these; or any combination thereof.

In some embodiments, an antifungal compound can be ciclopirox olamine; haloprogin; tolnaftate; undecylenate; topical nystatin; amorolfine; butenafine; naftifine; terbinafine; or any combination thereof.

In some aspects, a composition can comprise no more than about 10 mg, no more than about 20 mg, no more than about 40 mg, no more than about 60 mg, no more than about 80 mg, no more than about 100 mg, no more than about 120 mg, no more than about 140 mg, no more than about 160 mg, no more than about 180 mg, no more than about 200 mg, no more than about 300 mg, no more than about 400 mg, or no more than about 500 mg of an active agent or a salt thereof. In some cases, the composition can comprise at least about 10 mg, at least about 20 mg, at least about 40 mg, at least about 60 mg, at least about 80 mg, at least about 100 mg, at least about 120 mg, at least about 140 mg, at least about 160 mg, at least about 180 mg, at least about 200 mg, at least about 300 mg, at least about 400 mg, or at least about 500 mg of an active agent or a salt thereof.

An active agent can comprise a portion of the total weight of the composition. In some cases, an active agent can comprise from about 0.000001% to about 99%, from about 0.000001% to about 50%, from about 0.000001% to about 30%, from about 0.000001% to about 20%, from about 0.00005% to about 15%, from about 0.00005% to about 10%, from about 0.00001% to about 10%, from about 0.0001% to about 10%, or from about 0.0001% to about 5% by weight of the total weight of the composition.

In some embodiments, the composition can comprise alcohol at a concentration that allows the active agent to be solubilized. In some cases, the composition can have an alcohol concentration of at most about 10%, at most about 8%, at most about 6%, at most about 5%, at most about 4%, at most about 3%, at most about 2%, or at most about 1% by weight. In some cases, the alcohol concentration can be about 3.5% by weight.

In some embodiments, the composition can be substantially free of a surfactant. In some cases, a composition can comprise low amounts of surfactants. Surfactants can be non-ionic surfactants, cationic surfactants, amphoteric surfactants, or zwitterionic surfactants.

In some cases, a composition can have an surfactant concentration of at most about 10%, at most about 8%, at most about 6%, at most about 5%, at most about 4%, at most about 3%, at most about 2%, at most about 1%, or at most about 0.1% of a surfactant by weight of the total weight of the composition.

In the cases where a composition comprises an active agent or a salt thereof, the active agent or a salt thereof can be emulsified in the emulsion. When emulsified in the emulsion, the active agent can be in a form with an average particle size minimized to about the micrometer scale. In some embodiments, the average particle size can be minimized to about the nanometer scale. In some cases, the average particle size can be from about 0.001 nm to about 500 μm, from about 0.001 nm to about 400 μm, from about 0.001 nm to about 300 μm, from about 0.001 nm to about 200 μm, from about 0.001 nm to about 100 μm, from about 0.001 nm to about 90 μm, from about 0.001 nm to about 80 μm, from about 0.001 nm to about 70 μm, from about 0.001 nm to about 60 μm, from about 0.001 nm to about 50 μm, from about 0.001 nm to about 40 μm, from about 0.001 nm to about 30 μm, from about 0.001 nm to about 20 μm, from about 0.001 nm to about 10 μm, from about 0.001 nm to about 5 μm, from about 0.001 nm to about 1 μm, from about 0.001 nm to about 900 nm, from about 0.001 nm to about 800 nm, from about 0.001 nm to about 700 nm, from about 0.001 nm to about 600 nm, from about 0.001 nm to about 500 nm, from about 0.001 nm to about 400 nm, from about 0.001 nm to about 300 nm, from about 0.001 nm to about 200 nm, from about 0.001 nm to about 100 nm, from about 0.001 nm to about 90 nm, from about 0.001 nm to about 80 nm, from about 0.001 nm to about 70 nm, from about 0.001 nm to about 60 nm, from about 0.001 nm to about 50 nm, from about 0.001 nm to about 40 nm, from about 0.001 nm to about 30 nm, from about 0.001 nm to about 20 nm, from about 0.001 nm to about 10 nm, from about 0.001 nm to about 5 nm, from about 0.001 nm to about 1 nm, from about 0.001 nm to about 0.9 nm, from about 0.001 nm to about 0.8 nm, from about 0.001 nm to about 0.7 nm, from about 0.001 nm to about 0.6 nm, from about 0.001 nm to about 0.5 nm, from about 0.001 nm to about 0.4 nm, from about 0.001 nm to about 0.3 nm, from about 0.001 nm to about 0.2 nm, from about 0.001 nm to about 0.1 nm, from about 0.001 nm to about 0.09 nm, from about 0.001 nm to about 0.08 nm, from about 0.001 nm to about 0.07 nm, from about 0.001 nm to about 0.06 nm, from about 0.001 nm to about 0.05 nm, from about 0.001 nm to about 0.04 nm, from about 0.001 nm to about 0.03 nm, from about 0.001 nm to about 0.02 nm, or from about 0.001 nm to about 0.01 nm. In some embodiments, the average particle size can be about 0.01 nm, about 0.05 nm, about 0.1 nm, about 0.15 nm, about 0.2 nm, about 0.25 nm, about 0.3 nm, about 0.35 nm, about 0.4 nm, about 0.45 nm, about 0.5 nm, about 0.55 nm, about 0.6 nm, about 0.65 nm, about 0.7 nm, about 0.75 nm, about 0.8 nm, about 0.85 nm, about 0.9 nm, about 0.95 nm, about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1 μm, about 2 μm, about 3 μm, about 4 μm, about 5 μm, about 6 μm, about 7 μm, about 8 μm, about 9 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 150 μm, about 200 μm, about 250 μm, about 300 μm, about 350 μm, about 400 μm, about 450 μm, or about 500 μm.

### Methods of Treatment

The methods and compositions can be used for treating a condition or a disease. In some cases, a treating of a disease can include at least partially ameliorating at least one symptom of a disease or condition. Examples of a disease or condition that can be treated using a pharmaceutical composition as described herein can include an endometrial disorder, adenomyosis, a cancer, an inflammatory disorder, an infection, and any combination thereof. While exemplary diseases or conditions are recited herein, a person of ordinary skill in the art could use a pharmaceutical composition described herein to treat additional diseases or conditions by substituting additional active agents.

In some embodiments, a disease or condition can be an endometrial disorder. For example, an endometrial disorder may be endometriosis. Endometriosis can be an often painful disorder in which tissue that normally lines the inside of a uterus (e.g., the endometrium) grows outside the uterus (endometrial implant). The methods and compositions can be used for treating endometriosis that involves the ovaries, bowel or the tissue lining a pelvis or endometriosis in which the endometrial tissue spreads beyond a pelvic region. In endometriosis, displaced deposits break down and bleed with each menstrual cycle. Because this displaced tissue has no way to exit the body, it can become trapped. Surrounding tissue can become irritated, eventually developing scar tissue and adhesions-abnormal tissue that binds organs together. In some cases, treating the condition can comprise reducing an endometrial deposit to an amount that can be lower than prior to the treatment.

In some cases, an endometrial disorder can be adenomyosis. Adenomyosis is a disorder in which an inner lining of an endometrium breaks through the muscle wall of the uterus (the myometrium). Adenomyosis can cause menstrual cramps, lower abdominal pressure, and bloating before menstrual periods and can result in heavy periods. The condition can be located throughout the portions of a uterus or localized in one spot. Though the cause of adenomyosis isn't known, studies have suggested that various hormones—including estrogen, progesterone, prolactin, and follicle stimulating hormone—may trigger the condition. Current treatments can include an administering of an anti-inflammatory medication to reduce inflammation; administering of an intervention such as an aromatase inhibitor, a GnRH agonist, or a GnRH antagonist to suppress expression of hormones that can trigger the condition. In some cases, an incidence of adenomyosis and endometriosis can occur simultaneously.

Treatment of an endometrial disorder using a vaginal composition as described herein can be determined using a number of metrics known in the art. A treatment endpoint for an endometrial disorder can include an ability to become pregnant. In some cases, an ability to become pregnant can be determined using an in vitro assay, such as human chorionic gonadotropin (hCG) assay. An hCG assay can be performed on a sample from a subject, such as a blood sample. Such an assay could determine a level of hCG in the blood over time using an antibody specific for hCG, which can be used to determine an incidence of pregnancy. An ability to become pregnant can also be determined using an in vivo imaging means such as ultrasound to determine a presence of mature oocytes within the subject, or to determine a successful implantation of a zygote upon fertilization.

In some embodiments, a disease or condition can be a cancer. For example, the cancer can be cancer of the reproductive tract, cervical cancer, ovarian cancer, mesothelial cancer, peritoneal cancer, or any combination thereof.

In exemplary embodiments, administering vaginally a pharmaceutical composition described herein can be used to locally treat a cancer of the peritoneal cavity. Administering vaginally of a pharmaceutical composition described herein can be used to minimize adverse side effects that can occur through systemic administration. For example, a pharmaceutical composition comprising an antineoplastic as described above can be used to deliver the antineoplastic directly to the peritoneal cavity with only minimal delivery of the neoadjuvant into the circulatory system. As an exemplary illustration, such an administering vaginally of a pharmaceutical composition can be used to mitigate known side effects of systemic antineoplastic administration such as hair loss, reproductive side effects, skin or nail irritation, swelling, cardiotoxicity, hepatotoxicity, etc. Reduction of a side effect can be determined methods such as monitoring a lower incidence of a symptom of a side effect. Examples can include a reduction of local irritation or inflammation, a lower incidence of nausea, a lower incidence of pain, a reduction in irregular heart rate or arrhythmia, a decrease in overly frequent or painful urination, etc. Biomarkers can also be used to measure a decrease in a side effect. In some cases, a reduction of a side effect can include a reduction in a biomarker associated with toxicity. In some cases, a reduction of a side effect can include an increase in a biomarker associated with toxicity. Examples of biomarkers can include cardiac troponin I, cardiac troponin T, serum alanine aminotransferase, glutathione-S-transferase alpha, aspartate aminotransferase, serum creatinine, blood urea nitrogen, kidney injury molecule-1, eutrophil gelatinase-associated lipocalin (NGAL), interleukin-18 (IL-18), cystatin C, clusterin, fatty acid binding protein-liver type (L-FABP), osteopontin, etc.

In some cases, treating a cancer can comprise reducing reduce a tumor size, or slowing or preventing the growth of a tumor. A tumor burden of a subject can be determined using imaging techniques such as ultrasound or magnetic resonance imaging (MRI), which can be compared over time to determine a therapeutic effect of the administering of the pharmaceutical composition over time.

In some embodiments, the condition can be an inflammatory disorder. For example, the inflammatory disorder can be pelvic inflammatory disease, chromic pelvic pain, Other examples of inflammatory disease include sepsis, and chronic inflammation resulting from chronic viral or bacterial infection. In some cases, treating the condition can comprise reducing an amount of at least one proinflammatory cytokine to an amount that can be lower than prior to the treatment.

In some embodiments, the condition can be an infection. For example, the infection can be a bacterial infection, a viral infection, a fungal infection, or any combination thereof.

In some cases, an infection can include infection by a bacterial pathogen. A bacterial pathogen may be derived from a bacterial species selected from the group, but not exclusive to the group, consisting of: Staphylococcus spp., e.g. Staphylococcus aureus (e.g. Staphylococcus aureus NCTC 10442 and Staphylococcus aureus ATCC25923), Staphylococcus epidermidis; Chlamydia spp., e.g. Chlamydia trachomatis, Chlamydia pneumoniae, Chlamydia psittaci; Enterococcus spp., e.g. Enterococcus faecalis; Streptococcus pyogenes; Listeria spp.; Pseudomonas spp.; Mycobacterium spp., e.g. Mycobacterium tuberculosis complex; Enterobacter spp.; Campylobacter spp.; Salmonella spp.; Streptococcus spp., e.g. Streptococcus Group A or B, Streptoccocus pneumoniae; Helicobacter spp., e.g. Helicobacter pylori, Helicobacter felis; Neisseria spp., e.g. Neisseria gonorrhoea, Neisseria meningitidis; Borrelia burgdorferi; Shigella spp., e.g. Shigella flexneri; Escherichia coli (E. coli 0157: H7 NCTC 12900); Haemophilus spp., e.g. Haemophilus influenzae; Francisella tularensis; Bacillus spp., e.g. Bacillus anthraces; Clostridia spp., e.g. Clostridium botulinum, Clostridium difficile; Yersinia spp., e.g. Yersinia pestis; Treponema spp.; Burkholderia spp., e.g. Burkholderia cepacia complex, B. mallei, B pseudomallei; Propionibacterium spp., e.g. P. acnes, Acinetobacter species, an Actinomyces species, a Campylobacter species, a Candida species, Corynebacterium minutissium, Corynebacterium pseudodiphtheriae, Corynebacterium stratium, Corynebacterium group G1, Corynebacterium group G2, Enterobacteriaceae, an Enterococcus species, Klebsiella pneumoniae, a Moraxella species, a non-tuberculous mycobacteria species, a Porphyromonas species, Prevotella melaninogenicus, Salmonella typhimurium, Serratia marcescens Streptococcus agalactiae, Staphylococcus salivarius, Streptococcus mitis, Streptococcus sanguis, Streptococcus pneumoniae, Vibrio cholerae, a Coccidioides species, or a Cryptococcus species.

In some cases, an infection can include infection by a virus. A virus may be derived from the group, but not exclusive to the group, of a herpesvirus, a poxvirus, a hepadnavirus, a flavivirus, a togavirus, a coronavirus, hepatitis C, hepatitis D, an orthomyxovirus, a papillomavirus, a polyomaviridae, a parvovirus, a cytomegalovirus, an Epstein-Barr virus, a small pox virus, a cow pox virus, a sheep pox virus, an orf virus, a monkey pox virus, a vaccinia virus, a paramyxovirus, a retrovirus, an adenovirus, a rhabdovirus, a bunyavirus, a filovirus, an alphavirus, an arenavirus, a lentivirus, and any combination thereof. In some cases, the virus can be an enveloped virus. Examples of an enveloped viruses can include: a poxvirus, a hepadnavirus, a flavivirus, a togavirus, a coronavirus, hepatitis C, hepatitis D, an orthomyxovirus, a cytomegalovirus, an Epstein-Barr virus, a small pox virus, a cow pox virus, a sheep pox virus, an orf virus, a monkey pox virus, a vaccinia virus, a rhabdovirus, a bunyavirus, a filovirus, an alphavirus, an arenavirus, a lentivirus, and the like.

In some cases, an infection can include infection by a parasite selected from, but not limited to, the group consisting of Trypanosoma spp. (Trypanosoma cruzi, Trypansosoma brucei), Leishmania spp., Giardia spp., Trichomonas spp., Entamoeba spp., Naegleria spp., Acanthanioeba spp., Schistosoma spp., Plasmodium spp., Crytosporidium spp., Isospora spp., Balantidium spp., Loa, Ascaris lumbricoides, Dirofilaria immitis, and Toxoplasma ssp., e.g. Toxoplasma gondii.

A fungal pathogen may be derived from a fungus (including yeast) selected from, but not limited to, the genera Candida spp., (e.g. C.albicans), Epidermophyton spp., Exophiala spp., Microsporum spp., Trichophyton spp., (e.g. T. rubrum and T. interdigitale), Tinea spp., Aspergillus spp., Blastomyces spp., Blastoschizomyces spp., Coccidioides spp., Cryptococcus spp. (e.g. Cryptococcus neoformans), Histoplasma spp., Paracoccidiomyces spp., Sporotrix spp., Absidia spp., Cladophialophora spp., Fonsecaea spp., Phialophora spp., Lacazia spp., Arthrographis spp., Acremoniwn spp., Actinomadura spp., Apophysomyces spp., Emmonsia spp., Basidiobolus spp., Beauveria spp., Chrysosporium spp., Conidiobolus spp., Cunninghamella spp., Fusarium spp., Geotrichum spp., Graphiwn spp., Leptosphaeria spp., Malassezia spp. (e.g. Malassezia Furfur), Mucor spp., Neotestudina spp., Nocardia spp., Nocardiopsis spp., Paecilomyces spp., Phoma spp., Piedraia spp., Pneunwcystis spp., Pseudallescheria spp., Pyrenochaeta spp., Rhizoinucor spp., Rhizopus spp., Rhodotorula spp., Saccharomyces spp., Scedosporium spp., Scopulariopsis spp., Sporobolomyces spp., S: yncephalastrum spp., Trichoderma spp., Trichosporon spp., Ulocladium spp., Ustilago spp., Verticillium spp., and Wangiella spp.

It is also envisaged that a pharmaceutical composition described herein could be administered prophylactically in order to prevent incidence of a disease or condition described herein. For example, a pharmaceutical composition comprising an antibiotic could be administered vaginally to a subject prior to surgery to prevent a peritoneal infection.

### Dosing/Pharmacokinetics

In some embodiments, a pharmaceutical formulation can be formulated to optimize pharmacokinetics/pharmacodynamics of an active agent or salt thereof contained therein upon administering vaginally of a pharmaceutical composition to a subject.

In some cases, a pharmaceutical composition comprising an active agent or salt thereof described herein can be administered at a dose of from about 1 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg. In some cases, a pharmaceutical composition comprising an active agent or salt thereof described herein can be administered at a dose of from 0.1 mg up to about 200 mg. In some cases, a pharmaceutical composition comprising an active agent or salt thereof described herein can be administered at a dose of from 1 mg up to about 200 mg. In some cases, a pharmaceutical composition comprising an active agent or salt thereof described herein can be administered at a dose of from about 1 mg to 100 mg. In some cases, a pharmaceutical composition comprising an active agent or salt thereof described herein can be administered at a dose of from 1 mg up to about 100 mg.

In some cases, a pharmaceutical composition comprising an active agent or salt thereof described herein can be administered at a dose of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg. In some cases, the dose of the active agent or salt thereof is less than 600 mg, less than 500 mg, less than 400 mg, less than 300 mg, less than 200 mg, or about 100 mg. In some cases, a pharmaceutical composition comprising an active agent or salt thereof described herein can be administered at a dose of about 100 mg. In some cases, a pharmaceutical composition comprising an active agent or salt thereof described herein can be administered at a dose of about 200 mg. In some cases, a pharmaceutical composition comprising an active agent or salt thereof described herein can be administered at a dose of about 300 mg. In some cases, a pharmaceutical composition comprising an active agent or salt thereof described herein can be administered at a dose of about 400 mg. In some cases, a pharmaceutical composition comprising an active agent or salt thereof described herein can be administered at a dose of about 500 mg.

In some cases, a pharmaceutical composition comprising an active agent or salt thereof described herein can be administered to provide a blood plasma concentration of an active agent, a metabolite thereof, or salt thereof of from about 0.5 ng/mL to about 10 μg/mL, from about 1 ng/ml to about 10 μg/mL, from about 5 ng/ml to about 10 μg/mL, from about 10 ng/mL to about 10 μg/mL, from about 15 ng/ml to about 10 μg/mL, from about 20 ng/ml to about 10 μg/mL, from about 25 ng/ml to about 10 μg/mL, from about 30 ng/ml to about 10 μg/mL, from about 35 ng/ml to about 10 μg/mL, from about 40 ng/ml to about 10 μg/mL, from about 45 ng/ml to about 10 μg/mL, from about 50 ng/mL to about 10 μg/mL, from about 55 ng/ml to about 10 μg/mL, from about 60 ng/ml to about 10 μg/mL, from about 65 ng/mL to about 10 μg/mL, from about 70 ng/ml to about 10 μg/mL, from about 75 ng/ml to about 10 μg/mL, from about 80 ng/mL to about 10 μg/mL, from about 85 ng/mL to about 10 μg/mL, from about 90 ng/ml to about 10 μg/mL, from about 95 ng/ml to about 10 μg/mL, from about 100 ng/ml to about 10 μg/mL, from about 200 ng/ml to about 10 μg/mL, from about 300 ng/ml to about 10 μg/mL, from about 400 ng/mL to about 10 μg/mL, from about 500 ng/mL to about 10 μg/mL, from about 600 ng/ml to about 10 μg/mL, from about 700 ng/ml to about 10 μg/mL, from about 800 ng/ml to about 10 μg/mL, from about 900 ng/mL to about 10 μg/mL, or from about 1 μg/mL to about 10 μg/mL after a time period of from about 1 minute to about 1, 2, 3, 4, 5, 6, 7, or 10 or more hours. In some cases, a pharmaceutical composition comprising an active agent or salt thereof described herein can be administered to provide a blood plasma concentration of the active agent, a metabolite thereof, or salt thereof of from about 0.5 ng/mL to about 10 ng/mL.

In some cases a pharmaceutical composition comprising an active agent or salt thereof described herein can be administered to provide a blood plasma concentration of an active agent, a metabolite thereof, or salt thereof after administration to a subject of at least about 200 ng/mL, 195 ng/mL, 190 ng/mL, 185 ng/mL, 180 ng/mL, 175 ng/mL, 170 ng/mL, 165 ng/ml, 160 ng/mL, 155 ng/mL, 150 ng/mL, 145 ng/mL, 140 ng/mL, 135 ng/mL, 130 ng/mL, 125 ng/ml, 120 ng/mL, 115 ng/mL, 110 ng/mL, 105 ng/mL, 100 ng/ml, 95 ng/ml, 90 ng/ml, 85 ng/ml, 80 ng/mL, 75 ng/mL, 70 ng/mL, 65 ng/mL, 60 ng/mL, 55 ng/mL, 50 ng/mL, 45 ng/ml, 40 ng/ml, 35 ng/ml, 30 ng/ml, 25 ng/mL, 20 ng/mL, 15 ng/mL, 10 ng/mL, or 5 ng/ml after a time period of from about 1 minute to about 1, 2, 3, 4, 5, 6, 7, 8, 9, or 10 hours.

In some embodiments, vaginally administering a composition provided herein reduces systemic concentration of the active agent or the salt thereof in the subject by at least 10-fold relative to oral administration of the active agent or the salt thereof to a subject. In some embodiments, vaginally administering a composition provided herein reduces systemic concentration of the active agent or the salt thereof in the subject by at least 20-fold relative to oral administration of the active agent or the salt thereof to a subject. In some embodiments, vaginally administering a composition provided herein reduces systemic concentration of the active agent or the salt thereof in the subject by at least 30-fold relative to oral administration of the active agent or the salt thereof to a subject. In some embodiments, vaginally administering a composition provided herein reduces systemic concentration of the active agent or the salt thereof in the subject by at least 40-fold relative to oral administration of the active agent or the salt thereof to a subject. In some embodiments, a vaginal administration of a composition provided herein comprising about 100 mg of an active agent of a salt thereof reaches a serum concentration of about 1 ng/ml up to about 50 ng/mL. In some embodiments, a vaginal administration of a composition provided herein comprising about 100 mg of an active agent of a salt thereof reaches a serum concentration of about 0.1 ng/mL up to 50 ng/mL. In some embodiments, a vaginal administration of a composition provided herein comprising about 100 mg of an active agent of a salt thereof reaches a serum concentration of about 1 ng/mL up to about 10 ng/mL. In some embodiments, a pharmaceutical composition comprising 600 mg of the active agent or a salt thereof when orally administered to a subject, reaches a serum concentration of about 100 ng/ml up to about 500 ng/mL.

In some cases a pharmaceutical composition comprising an active agent or salt thereof described herein can be administered to provide a concentration of an active agent, a metabolite thereof, or salt thereof in a peritoneal fluid after administration to a subject of at least about 200 ng/mL, 195 ng/mL, 190 ng/mL, 185 ng/mL, 180 ng/mL, 175 ng/mL, 170 ng/mL, 165 ng/mL, 160 ng/mL, 155 ng/mL, 150 ng/mL, 145 ng/mL, 140 ng/mL, 135 ng/mL, 130 ng/ml, 125 ng/mL, 120 ng/mL, 115 ng/mL, 110 ng/ml, 105 ng/ml, 100 ng/ml, 95 ng/ml, 90 ng/mL, 85 ng/mL, 80 ng/mL, 75 ng/mL, 70 ng/mL, 65 ng/mL, 60 ng/mL, 55 ng/mL, 50 ng/ml, 45 ng/mL, 40 ng/mL, 35 ng/mL, 30 ng/mL, 25 ng/mL, 20 ng/mL, 15 ng/mL, 10 ng/ml, 9 ng/ml, 8 ng/ml, 7 ng/mL, 6 ng/ml, 5 ng/mL, 4 ng/mL, 3 ng/mL, 2 ng/mL, 1 ng/mL, 0.9 ng/ml, 0.8 ng/mL, 0.7 ng/mL, 0.6 ng/mL, 0.5 ng/mL, 0.4 ng/mL, 0.3 ng/mL, 0.2 ng/mL, or 0.1 ng/mL after a time period of from about 1 minute to about 1, 2, 3, 4, 5, 6, 7, 8, 9, or 10 hours.

In some cases a pharmaceutical composition comprising an active agent or salt thereof described herein can be administered to provide a concentration of an active agent, a metabolite thereof, or salt thereof in a peritoneal fluid after administration to a subject of from about 0.5 ng/mL to about 100 ng/ml, from about 0.5 ng/mL to about 90 ng/ml, from about 0.5 ng/ml to about 80 ng/mL, from about 0.5 ng/ml to about 70 ng/ml, from about 0.5 ng/ml to about 60 ng/mL, from about 0.5 ng/mL to about 50 ng/mL, from about 0.5 ng/ml to about 40 ng/ml, from about 0.5 ng/ml to about 30 ng/mL, from about 0.5 ng/ml to about 20 ng/ml, from about 0.5 ng/ml to about 10 ng/mL, from about 0.5 ng/ml to about 9 ng/mL, from about 0.5 ng/ml to about 8 ng/mL, from about 0.5 ng/ml to about 7 ng/ml, from about 0.5 ng/ml to about 6 ng/mL, from about 0.5 ng/mL to about 5 ng/mL, from about 0.5 ng/mL to about 4 ng/ml, from about 0.5 ng/ml to about 3 ng/ml, from about 0.5 ng/ml to about 2 ng/ml, or from about 0.5 ng/ml to about 1 ng/ml after a time period of from about 1 minute to about 1, 2, 3, 4, 5, 6, 7, 8, 9, or 10 hours.

In some cases a pharmaceutical composition comprising an active agent or salt thereof described herein can be administered to provide a concentration of an active agent, a metabolite thereof, or salt thereof in a peritoneal tissue after administration to a subject of at least about 200 ng/mg, 195 ng/mg, 190 ng/mg, 185 ng/mg, 180 ng/mg, 175 ng/mg, 170 ng/mg, 165 ng/mg, 160 ng/mg, 155 ng/mg, 150 ng/mg, 145 ng/mg, 140 ng/mg, 135 ng/mg, 130 ng/mg, 125 ng/mg, 120 ng/mg, 115 ng/mg, 110 ng/mg, 105 ng/mg, 100 ng/mg, 95 ng/mg, 90 ng/mg, 85 ng/mg, 80 ng/mg, 75 ng/mg, 70 ng/mg, 65 ng/mg, 60 ng/mg, 55 ng/mg, 50 ng/mg, 45 ng/mg, 40 ng/mg, 35 ng/mg, 30 ng/mg, 25 ng/mg, 20 ng/mg, 15 ng/mg, 10 ng/mg, 9 ng/mg, 8 ng/mg, 7 ng/mg, 6 ng/mg, 5 ng/mg, 4 ng/mg, 3 ng/mg, 2 ng/mg, 1 ng/mg, 0.9 ng/mg, 0.8 ng/mg, 0.7 ng/mg, 0.6 ng/mg, 0.5 ng/mg, 0.4 ng/mg, 0.3 ng/mg, 0.2 ng/mg, or 0.1 ng/mg after a time period of from about 1 minute to about 1, 2, 3, 4, 5, 6, 7, 8, 9, or 10 hours.

In some cases a pharmaceutical composition comprising an active agent or salt thereof described herein can be administered to provide a concentration of an active agent, a metabolite thereof, or salt thereof in a peritoneal tissue after administration to a subject of from about 0.5 ng/mg to about 100 ng/mg, from about 0.5 ng/mg to about 90 ng/mg, from about 0.5 ng/mg to about 80 ng/mg, from about 0.5 ng/mg to about 70 ng/mg, from about 0.5 ng/mg to about 60 ng/mg, from about 0.5 ng/mg to about 50 ng/mg, from about 0.5 ng/mg to about 40 ng/mg, from about 0.5 ng/mg to about 30 ng/mg, from about 0.5 ng/mg to about 20 ng/mg, from about 0.5 ng/mg to about 10 ng/mg, from about 0.5 ng/mg to about 9 ng/mg, from about 0.5 ng/mg to about 8 ng/mg, from about 0.5 ng/mg to about 7 ng/mg, from about 0.5 ng/mg to about 6 ng/mg, from about 0.5 ng/mg to about 5 ng/mg, from about 0.5 ng/mg to about 4 ng/mg, from about 0.5 ng/mg to about 3 ng/mg, from about 0.5 ng/mg to about 2 ng/mg, or from about 0.5 ng/mg to about 1 ng/mg after a time period of from about 1 minute to about 1, 2, 3, 4, 5, 6,7, 8, 9, or 10 hours.

In some cases, a pharmaceutical composition comprising an active agent or salt thereof described herein can be administered to provide a Tmax of an active agent or salt thereof after administration to a subject of from about 1 minute to about 600 minutes, from about 1 minute to about 590 minutes, from about 1 minute to about 580 minutes, from about 1 minute to about 570 minutes, from about 1 minute to about 560 minutes, from about 1 minute to about 550 minutes, from about 1 minute to about 540 minutes, from about 1 minute to about 530 minutes, from about 1 minute to about 520 minutes, from about 1 minute to about 510 minutes, from about 1 minute to about 500 minutes, from about 1 minute to about 490 minutes, from about 1 minute to about 480 minutes, from about 1 minute to about 470 minutes, from about 1 minute to about 460 minutes, from about 1 minute to about 450 minutes, from about 1 minute to about 440 minutes, from about 1 minute to about 430 minutes, from about 1 minute to about 420 minutes, from about 1 minute to about 410 minutes, from about 1 minute to about 400 minutes, from about 1 minute to about 390 minutes, from about 1 minute to about 380 minutes, from about 1 minute to about 370 minutes, from about 1 minute to about 360 minutes, from about 1 minute to about 350 minutes, from about 1 minute to about 340 minutes, from about 1 minute to about 330 minutes, from about 1 minute to about 320 minutes, from about 1 minute to about 310 minutes, from about 1 minute to about 300 minutes, from about 1 minute to about 290 minutes, from about 1 minute to about 280 minutes, from about 1 minute to about 270 minutes, from about 1 minute to about 260 minutes, from about 1 minute to about 250 minutes, from about 1 minute to about 240 minutes, from about 1 minute to about 230 minutes, from about 1 minute to about 220 minutes, from about 1 minute to about 210 minutes, from about 1 minute to about 200 minutes, from about 1 minute to about 190 minutes, from about 1 minute to about 180 minutes, from about 1 minute to about 170 minutes, from about 1 minute to about 160 minutes, from about 1 minute to about 150 minutes, from about 1 minute to about 140 minutes, from about 1 minute to about 130 minutes, from about 1 minute to about 120 minutes, from about 1 minute to about 110 minutes, from about 1 minute to about 100 minutes, from about 1 minute to about 90 minutes, from about 1 minute to about 80 minutes, from about 1 minute to about 70 minutes, from about 1 minute to about 60 minutes, from about 1 minute to about 50 minutes, from about 1 minute to about 40 minutes, from about 1 minute to about 30 minutes, from about 1 minute to about 20 minutes, from about 1 minute to about 10 minutes, from about 1 minute to about 9 minutes, from about 1 minute to about 8 minutes, from about 1 minute to about 7 minutes, from about 1 minute to about 6 minutes, from about 1 minute to about 5 minutes, from about 1 minute to about 4 minutes, from about 1 minute to about 3 minutes, or from about 1 minute to about 2 minutes.

In some cases, a pharmaceutical composition comprising an active agent or salt thereof described herein can be administered to provide a Tmax of an active agent, a metabolite thereof, or salt thereof after administration to a subject of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, or 200 minutes. In some cases, a pharmaceutical composition comprising an active agent or salt thereof described herein can be administered to provide a Tmax of an active agent, a metabolite thereof, or salt thereof after administration to a subject of at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0 hours.

In some cases, a pharmaceutical composition comprising an active agent or salt thereof described herein can be administered to provide a Cmax of an active agent, a metabolite thereof, or salt thereof after administration to a subject of at least about 1,000 μg/mL, 950 μg/mL, 900 μg/mL, 850 μg/mL, 800 μg/mL, 750 μg/mL, 700 μg/mL, 650 μg/mL, 600 μg/mL, 550 μg/mL, 500 μg/mL, 450 μg/mL, 400 μg/mL, 350 μg/mL, 300 μg/mL, 250 μg/mL, 200 μg/mL, 150 μg/mL, 100 μg/mL, or 50 μg/mL. In some cases, a pharmaceutical composition comprising an active agent or salt thereof described herein can be administered to provide a Cmax of an active agent, a metabolite thereof, or salt thereof after administration to a subject of at least about 100 μg/mL, 95 μg/mL, 90 μg/mL, 85 μg/mL, 80 μg/mL, 75 μg/mL, 70 μg/mL, 65 μg/mL, 60 μg/mL, 55 μg/mL, 50 μg/mL, 45 μg/mL, 40 μg/mL, 35 μg/mL, 30 μg/mL, 25 μg/mL, 20 μg/mL, 15 μg/mL, 10 μg/mL, 5 μg/mL, 4 μg/mL, 3 μg/mL, 2 μg/mL, or 1 μg/mL. In some cases, an active agent, salt thereof, or pharmaceutical composition comprising an active agent or salt thereof described herein can be administered to provide a Cmax of an active agent, a metabolite thereof, or salt thereof after administration to a subject of at least about 1,000 ng/ml, 950 ng/mL, 900 ng/ml, 850 ng/ml, 800 ng/mL, 750 ng/mL, 700 ng/mL, 650 ng/mL, 600 ng/mL, 550 ng/mL, 500 ng/ml, 450 ng/ml, 400 ng/ml, 350 ng/mL, 300 ng/mL, 250 ng/mL, 200 ng/mL, 150 ng/mL, 100 ng/mL, or 50 ng/ml. In some cases, a pharmaceutical composition comprising an active agent or salt thereof described herein can be administered to provide a Cmax of an active agent, a metabolite thereof, or salt thereof after administration to a subject of at least about 100 ng/ml, 95 ng/ml, 90 ng/ml, 85 ng/mL, 80 ng/mL, 75 ng/mL, 70 ng/mL, 65 ng/mL, 60 ng/mL, 55 ng/mL, 50 ng/ml, 45 ng/mL, 40 ng/mL, 35 ng/mL, 30 ng/mL, 25 ng/mL, 20 ng/mL, 15 ng/mL, 10 ng/ml, or 5 ng/ml. In some cases, a pharmaceutical composition comprising an active agent or salt thereof described herein can be administered to provide a Cmax of an active agent, a metabolite thereof, or salt thereof of at least about 50 ng/mL, 49 ng/mL, 48 ng/mL, 47 ng/mL, 46 ng/mL, 45 ng/ml, 44 ng/mL, 43 ng/ml, 42 ng/mL, 41 ng/mL, 40 ng/mL, 39 ng/mL, 38 ng/mL, 37 ng/mL, 36 ng/mL, 35 ng/mL, 34 ng/ml, 33 ng/mL, 32 ng/mL, 31 ng/ml, 30 ng/mL, 29 ng/mL, 28 ng/ml, 27 ng/ml, 26 ng/ml, 25 ng/ml, 24 ng/mL, 23 ng/mL, 22 ng/mL, 21 ng/mL, 20 ng/mL, 19 ng/mL, 18 ng/mL, 17 ng/mL, 16 ng/ml, 15 ng/ml, 14 ng/ml, 13 ng/mL, 12 ng/mL, 11 ng/ml, 10 ng/ml, 9 ng/ml, 8 ng/ml, 7 ng/ml, 6 ng/mL, 5 ng/mL, 4 ng/ml, 3 ng/ml, 2 ng/ml, 1 ng/mL, or 0.5 ng/mL.

In some embodiments, a pharmaceutical composition comprising an active agent or salt thereof described herein can be administered to provide an AUC (0-t) of an active agent, a metabolite thereof, or salt thereof after administration to a subject of at least about 10,000 ng*h/mL, 9,900 ng*h/mL, 9,800 ng*h/mL, 9,700 ng*h/mL, 9,600 ng*h/mL, 9,500 ng*h/mL, 9,400 ng*h/mL, 9,300 ng*h/mL, 9,200 ng*h/mL, 9,100 ng*h/mL, 9,000 ng*h/mL, 8,900 ng*h/mL, 8,800 ng*h/mL, 8,700 ng*h/mL, 8,600 ng*h/mL, 8,500 ng*h/mL, 8,400 ng*h/mL, 8,300 ng*h/mL, 8,200 ng*h/mL, 8,100 ng*h/mL, 8,000 ng*h/mL, 7,900 ng*h/mL, 7,800 ng*h/mL, 7,700 ng*h/mL, 7,600 ng*h/mL, 7,500 ng*h/mL, 7,400 ng*h/mL, 7,300 ng*h/mL, 7,200 ng*h/mL, 7,100 ng*h/mL, 7,000 ng*h/mL, 6,900 ng*h/mL, 6,800 ng*h/mL, 6,700 ng*h/mL, 6,600 ng*h/mL, 6,500 ng*h/mL, 6,400 ng*h/mL, 6,300 ng*h/mL, 6,200 ng*h/mL, 6,100 ng*h/mL, 6,000 ng*h/mL, 5,900 ng*h/mL, 5,800 ng*h/mL, 5,700 ng*h/mL, 5,600 ng*h/mL, 5,500 ng*h/mL, 5,400 ng*h/mL, 5,300 ng*h/mL, 5,200 ng*h/mL, 5,100 ng*h/mL, 5,000 ng*h/mL, 4,500 ng*h/mL, 4,000 ng*h/mL, 3,500 ng*h/mL, 3,000 ng*h/mL, 2,500 ng*h/mL, 2,000 ng*h/mL, 1,500 ng*h/mL, or 1,900 ng*h/mL, where it can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 hours after administration of a pharmaceutical composition comprising an active agent or salt thereof.

In some embodiments, a pharmaceutical composition comprising an active agent or salt thereof described herein can be administered to provide an AUC (0-f) of a an active agent, a metabolite thereof, or salt thereof after administration to a subject of at least about 10,000 ng*h/mL, 9,900 ng*h/mL, 9,800 ng*h/mL, 9,700 ng*h/mL, 9,600 ng*h/mL, 9,500 ng*h/mL, 9,400 ng*h/mL, 9,300 ng*h/mL, 9,200 ng*h/mL, 9,100 ng*h/mL, 9,000 ng*h/mL, 8,900 ng*h/mL, 8,800 ng*h/mL, 8,700 ng*h/mL, 8,600 ng*h/mL, 8,500 ng*h/mL, 8,400 ng*h/mL, 8,300 ng*h/mL, 8,200 ng*h/mL, 8,100 ng*h/mL, 8,000 ng*h/mL, 7,900 ng*h/mL, 7,800 ng*h/mL, 7,700 ng*h/mL, 7,600 ng*h/mL, 7,500 ng*h/mL, 7,400 ng*h/mL, 7,300 ng*h/mL, 7,200 ng*h/mL, 7,100 ng*h/mL, 7,000 ng*h/mL, 6,900 ng*h/mL, 6,800 ng*h/mL, 6,700 ng*h/mL, 6,600 ng*h/mL, 6,500 ng*h/mL, 6,400 ng*h/mL, 6,300 ng*h/mL, 6,200 ng*h/mL, 6,100 ng*h/mL, 6,000 ng*h/mL, 5,900 ng*h/mL, 5,800 ng*h/mL, 5,700 ng*h/mL, 5,600 ng*h/mL, 5,500 ng*h/mL, 5,400 ng*h/mL, 5,300 ng*h/mL, 5,200 ng*h/mL, 5,100 ng*h/mL, 5,000 ng*h/mL, 4,500 ng*h/mL, 4,000 ng*h/mL, 3,500 ng*h/mL, 3,000 ng*h/mL, 2,500 ng*h/mL, 2,000 ng*h/mL, 1,500 ng*h/mL, or 1,900 ng*h/mL, where t can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days after administration of a pharmaceutical composition comprising an active agent or salt thereof.

In some exemplary embodiments, a pharmaceutical composition comprising an active agent or salt thereof described herein can be administered to provide an AUC (0-t) of an active agent, a metabolite thereof, or salt thereof after administration to a subject of from about 1,000 ng*h/mL to about 10,000 ng*h/mL, from about 1,000 ng*h/mL to about 9,000 ng*h/mL, from about 1,000 ng*h/mL to about 8,000 ng*h/mL, from about 1,000 ng*h/mL to about 7,000 ng*h/mL, from about 1,000 ng*h/mL to about 6,000 ng*h/mL, from about 1,000 ng*h/mL to about 5,000 ng*h/mL, from about 1,000 ng*h/mL to about 4,000 ng*h/mL, from about 1,000 ng*h/mL to about 3,000 ng*h/mL, or from about 1,000 ng*h/mL to about 2,000 ng*h/mL, where t can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days after administration of a pharmaceutical composition comprising an active agent or salt thereof.

In some exemplary embodiments, a pharmaceutical formulation can be produced such that when a pharmaceutical formulation is administered to a primate, an active agent or salt thereof can have a Tₘₐₓ of from about 1 minute to about 1 hour, a Cₘₐₓ of from about 1 minute to about 8 hours, an AUC_{0>24} hour of from about 0.1 μg·hr/L to about 1,000 μg·hr/L, a half-life of from about 2 hours to about 24 hours, or a combination thereof.

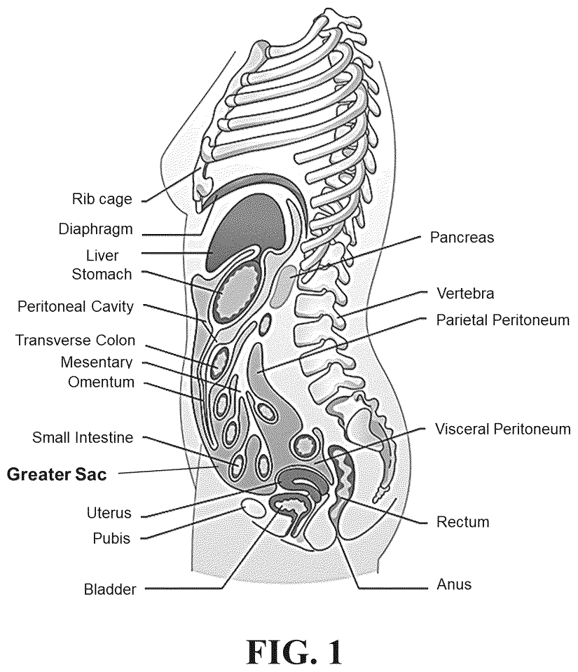

In some embodiments, a pharmaceutical formulation can be formulated such that, when a pharmaceutical formulation is administered to a subject, an active agent or salt thereof can be substantially localized in a peritoneal organ or tissue of a subject. Examples of organs or tissues of the peritoneal cavity can include those depicted in FIG. 1. A peritoneal cavity can include peritoneal fluid. A peritoneal organ or tissue can include, but is not limited to: a bladder, a gall bladder, an intestine, a uterus, an endometrium, a myometrium, a perimetrium, a stomach, an ovary, an ovarian cortex, an ovarian epithelium, a liver, a spleen, or a kidney.

In some embodiments, when a pharmaceutical formulation is administered to a subject, an active agent can have a half-life of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, or 200 minutes. In some embodiments, when a pharmaceutical formulation is administered to a subject, an active agent or salt thereof can have a half-life of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0 hours. In some embodiments, when a pharmaceutical formulation is administered to a subject, an active agent or salt thereof can have a half-life of from about 1 minute to about 600 minutes, from about 1 minute to about 590 minutes, from about 1 minute to about 580 minutes, from about 1 minute to about 570 minutes, from about 1 minute to about 560 minutes, from about 1 minute to about 550 minutes, from about 1 minute to about 540 minutes, from about 1 minute to about 530 minutes, from about 1 minute to about 520 minutes, from about 1 minute to about 510 minutes, from about 1 minute to about 500 minutes, from about 1 minute to about 490 minutes, from about 1 minute to about 480 minutes, from about 1 minute to about 470 minutes, from about 1 minute to about 460 minutes, from about 1 minute to about 450 minutes, from about 1 minute to about 440 minutes, from about 1 minute to about 430 minutes, from about 1 minute to about 420 minutes, from about 1 minute to about 410 minutes, from about 1 minute to about 400 minutes, from about 1 minute to about 390 minutes, from about 1 minute to about 380 minutes, from about 1 minute to about 370 minutes, from about 1 minute to about 360 minutes, from about 1 minute to about 350 minutes, from about 1 minute to about 340 minutes, from about 1 minute to about 330 minutes, from about 1 minute to about 320 minutes, from about 1 minute to about 310 minutes, from about 1 minute to about 300 minutes, from about 1 minute to about 290 minutes, from about 1 minute to about 280 minutes, from about 1 minute to about 270 minutes, from about 1 minute to about 260 minutes, from about 1 minute to about 250 minutes, from about 1 minute to about 240 minutes, from about 1 minute to about 230 minutes, from about 1 minute to about 220 minutes, from about 1 minute to about 210 minutes, from about 1 minute to about 200 minutes, from about 1 minute to about 190 minutes, from about 1 minute to about 180 minutes, from about 1 minute to about 170 minutes, from about 1 minute to about 160 minutes, from about 1 minute to about 150 minutes, from about 1 minute to about 140 minutes, from about 1 minute to about 130 minutes, from about 1 minute to about 120 minutes, from about 1 minute to about 110 minutes, from about 1 minute to about 100 minutes, from about 1 minute to about 90 minutes, from about 1 minute to about 80 minutes, from about 1 minute to about 70 minutes, from about 1 minute to about 60 minutes, from about 1 minute to about 50 minutes, from about 1 minute to about 40 minutes, from about 1 minute to about 30 minutes, from about 1 minute to about 20 minutes, from about 1 minute to about 10 minutes, from about 1 minute to about 9 minutes, from about 1 minute to about 8 minutes, from about 1 minute to about 7 minutes, from about 1 minute to about 6 minutes, from about 1 minute to about 5 minutes, from about 1 minute to about 4 minutes, from about 1 minute to about 3 minutes, or from about 1 minute to about 2 minutes.

### Methods of Detecting an Agent

Provided herein are methods of detecting an active agent, a metabolite thereof, or a salt thereof in a sample from a subject after an administering of a pharmaceutical composition to a subject. A sample from a subject may be blood or any excretory liquid. Non-limiting examples of may include saliva, blood, serum, cerebrospinal fluid, semen, feces, plasma, or urine.

In some exemplary embodiments, the method can be a method of detecting danazol or a salt thereof in a sample from a subject. A method can comprise contacting a portion of a sample from a subject with an albumin; and detecting danazol or a salt thereof by mass spectrometry. An albumin can include a human or bovine albumin. In some cases, the albumin can be a serum albumin.

Detection of an active agent such as danazol can be carried out by contacting the active agent with an albumin to form an albumin adduct and determining a presence of the adduct using mass spectrometry. A method can further comprise comparison to an internal standard. An exemplary internal standard can include 19-Norethindrone.

A mass spectrometer can include a single or tandem mass spectrometer. A mass spectrometer can comprise an electrospray ionizer, a matrix-assisted laser desorption/ionization ionizer, an electron ionizer, a fast atom bombardment ionizer or a chemical ionizer.

An exemplary method can comprise detecting danazol by determining an amount of an [M+H]⁺ ion. In some cases, an [M+H]⁺ ion can comprise an m/z of about 338.

In some embodiments, a sample can be dried after contacting with an albumin. In some embodiments, a sample can be reconstituted, resuspended, or diluted in a resuspension buffer after contacting with an albumin. A resuspension buffer can a buffering agent such as saline, citrate, phosphate, phosphate buffered saline, acetate, glycine, tris(hydroxymethyl)aminomethane (tris) hydrochloride, tris buffered saline (TBS), 3-[[1,3-dihydroxy-2-(hydroxymethyl) propan-2-yl]amino]propane-1-sulfonic acid (TAPS), bicine, tricine, 3-[1,3-dihydroxy-2-(hydroxymethyl) propan-2-yl]amino]-2-hydroxypropane-1-sulfonic acid (TAPSO), 4-(2-hydroxyethyl)-1-piperazineethanesulfonic acid (HEPES), piperazine-N,N′-bis(2-ethanesulfonic acid) (PIPES), 3-(N-morpholino) propanesulfonic acid (MOPS), 2-(N-morpholino) ethanesulfonic acid (MES), 2-[[1,3-dihydroxy-2-(hydroxymethyl) propan-2-yl]amino]ethanesulfonic acid (TES), cacodylate, glycine, carbonate, or any combination thereof.

### Kits

Disclosed herein are kits. In some aspects, a composition can be packaged in a container. In some aspects, a kit can further comprise instructions that direct administration of a unit dose of a composition to a subject.

Methods of making a kit can include placing a pharmaceutical composition in a container. A method can further comprise an inclusion of instructions for use. In some cases, instructions for use can direct administration of a unit dose of a composition to a subject.

Provided herein are kits for determining an amount of danazol in a sample. A kit can comprise: a sample collection vessel; an albumin; and instructions for use. In some cases, an internal standard can be 19-Norethindrone or a salt thereof. Instructions for use can direct a user to: collect a sample in the sample collection vessel; contact a portion of the sample with an amount of the albumin; and detect danazol or a salt thereof by mass spectrometry. In some cases, a kit can further comprise an internal standard. An internal standard can be 19-Norethindrone or a salt thereof.

### Methods of Treatment

Provided herein are methods of treating a disease in a subject, the methods comprising: administering to the subject a composition provided herein. In some embodiments, the composition comprises danazol or a salt thereof. In some embodiments, the subject has, is suspected of having, is diagnosed with, or is at risk of developing an endometrial disease, an infection, a cancer, dysmenorrhea, dyspareunia, pelvic pain, rectovaginal plaques, infertility, pre-menopause, menopause, or a hormonal imbalance.

Further provided herein are methods of treating an endometrial disease or disorder in a subject in need thereof, the methods comprising administering to a subject in need thereof a composition provided herein. In some embodiments, the subject has a menstrual cycle. In some embodiments, the subject has an endometrial lesion. In some embodiments, the subject has had one or more incidence of infertility or a miscarriage.

Endometriosis is a condition where endometrial tissue is found outside the uterus. It is ‘trapped’ in the pelvic area and abdomen and, rarely, in other areas in the body. Symptoms of endometriosis include, for example, painful periods, painful sex, pelvic pain, reduced fertility, pain on passing feces, pain in the lower abdomen when passing urine, blood in the urine or feces. Rarely, but in some cases, patches of endometriosis occur in other sites of the body. This can cause unusual pains in parts of the body that occur at the same time as period pains.

Further provided herein are methods of treating a disease in a subject, the methods comprising: administering a composition provided herein to the subject, wherein the composition comprises two or more agents provided herein. In some embodiments, the composition comprises: (1) danazol, a derivative, or a salt thereof; and (2) estrogen, a derivative, or a salt thereof.

### Exemplary Embodiments

Provided herein are compositions comprising a cream, a polybioadhesive gel, or a liquid emulsion provided herein.

Provided herein are creams, wherein the creams comprise: (a) micronized danazol or a salt thereof that is present in an amount of 5.0% up to about 7.0% weight to volume (w/v) of the cream; (b) polyglycolyzed oleic glyceride or a mixture thereof that is present in an amount of 10% up to 20% (w/v) of the cream; (c) a cellulose polymer that is present in an amount of 1.0% up to 3.0% (w/v) of the cream; and (d) one or more water-insoluble bioadhesives, wherein at least one water-insoluble bioadhesive is polycarbophil that is present in an amount of 1.0% up to about 2.0% (w/v) of the cream, wherein the cream comprises at least 60% (w/v) water and comprises one or more ingredients selected from: a carbomer, methylparaben, sorbic acid, a poloxamer, and a sodium carboxymethylcellulose. Further provided herein are creams, wherein the cream comprises the carbomer, wherein the carbomer is present in an amount of 0.1% up to 2.0% (w/v) of the cream. Further provided herein are creams, wherein the water-insoluble bioadhesive is dispersed or suspended in an aqueous phase of the cream. Further provided herein are creams, wherein the creams are in the form of a liquid emulsion. Further provided herein are creams, wherein the cream comprises at least 70% water. Further provided herein are creams, wherein the cream comprises the sorbic acid, wherein the sorbic acid is present in an amount of 0.1% up to 2.0% (w/v) of the cream. Further provided herein are creams, wherein the micronized danazol or the salt thereof is dispersed or suspended in an oily phase of the cream. Further provided herein are creams, wherein the dose of micronized danazol or the salt thereof is between 50 milligrams and 200 milligrams. Further provided herein are creams, wherein the cream is formulated for vaginal administration. Further provided herein are creams, wherein upon vaginal administration of the cream, the active agent or the salt thereof diffuses into the peritoneal fluid, reaching further pelvic tissue via transvaginal absorption and the counter-current transfer between uterovaginal lymph vessels or veins and arteries localizing the active agent to the peritoneal cavity.

Provided herein are compositions, wherein the compositions comprise: a polybioadhesive gel comprising: (a) an active agent or a salt thereof; (b) one or more water-insoluble bioadhesive; (c) at least one oleogel; and (d) at least one aqueous gel, wherein upon administration to a subject, the active agent localizes to the peritoneal tissue and induces pelvic pass metabolism in the subject. Further provided herein are compositions, wherein the active agent is a synthetic steroid. Further provided herein are compositions, wherein the synthetic steroid is danazol or a salt thereof. Further provided herein are compositions, wherein the polybioadhesive gel is formulated as a liquid emulsion. Further provided herein are compositions, wherein the compositions are formulated for vaginal administration. Further provided herein are compositions, wherein upon vaginal administration of the composition, the active agent or the salt thereof diffuses into the peritoneal fluid, reaching further pelvic tissue via transvaginal absorption and the counter-current transfer between uterovaginal lymph vessels or veins and arteries localizing the active agent to the peritoneal cavity.

Provided herein are compositions, wherein the compositions comprise: the components of Table 3 and a respective quantity (w/v) of each component. Further provided herein are compositions, wherein the compositions are formulated for vaginal administration. Further provided herein are compositions, wherein upon vaginal administration of the composition, the active agent or the salt thereof diffuses into the peritoneal fluid, reaching further pelvic tissue via transvaginal absorption and the counter-current transfer between uterovaginal lymph vessels or veins and arteries localizing the active agent to the peritoneal cavity.

Provided herein are methods for treating an endometrial disease or condition in a subject, wherein the methods comprise: vaginally administering to the subject a polybioadhesive gel comprising: (a) an active agent or a salt thereof; (b) one or more water-insoluble bioadhesives; (c) at least one oleogel; and (d) at least one aqueous gel, wherein vaginally administering the polybioadhesive gel localizes the active agent to the peritoneal tissue, thereby treating the endometrial disease or condition. Further provided herein are methods, wherein vaginally administering the polybioadhesive gel reduces the systemic concentration of the active agent or the salt thereof relative to a comparable oral composition comprising the same active agent. Further provided herein are methods, wherein the vaginally administering is performed once every 24 hours. Further provided herein are methods, wherein the active agent is a synthetic steroid. Further provided herein are methods, wherein the synthetic steroid is danazol or a salt thereof. Further provided herein are methods, wherein the active agent is micronized. Further provided herein are methods, wherein the subject has or is suspected of having infertility. Further provided herein are methods, wherein the subject has or is suspected of having endometriosis. Further provided herein are methods, wherein the vaginally administering reduces the number of endometrial lesions in the subject by at least 10% relative to the number of endometrial lesions in the subject before vaginally administering the polybioadhesive gel. Further provided herein are methods, wherein the vaginally administering reduces the size of one or more endometrial lesions in the subject by at least 10% relative to the size of one or more endometrial lesions prior to vaginally administering the polybioadhesive gel. Further provided herein are methods, wherein the polybioadhesive gel is formulated as a liquid emulsion. Further provided herein are methods, wherein the endometrial disorder is endometriosis, adenomyosis, or a combination thereof. Further provided herein are methods, wherein the polybioadhesive gel does not disrupt ovarian function in the subject. Further provided herein are methods, wherein the polybioadhesive gel reduces pain in the subject by at least 10% relative to pain identified by the subject prior to vaginally administering the polybioadhesive gel. Further provided herein are methods, wherein vaginally administering a composition provided herein reduces systemic concentration of the active agent or the salt thereof in the subject by at least 10-fold relative to oral administration of the active agent or the salt thereof to a subject. Further provided herein are methods, wherein vaginally administering a composition provided herein reduces systemic concentration of the active agent or the salt thereof in the subject by at least 15-fold relative to oral administration of the active agent or the salt thereof to a subject. Further provided herein are methods, wherein vaginally administering a composition provided herein reduces systemic concentration of the active agent or the salt thereof in the subject by at least 20-fold relative to oral administration of the active agent or the salt thereof to a subject. Further provided herein are methods, wherein vaginally administering a composition provided herein reduces systemic concentration of the active agent or the salt thereof in the subject by at least 25-fold relative to oral administration of the active agent or the salt thereof to a subject. Further provided herein are methods, wherein vaginally administering a composition provided herein reduces systemic concentration of the active agent or the salt thereof in the subject by at least 30-fold relative to oral administration of the active agent or the salt thereof to a subject. Further provided herein are methods, wherein vaginally administering a composition provided herein reduces systemic concentration of the active agent or the salt thereof in the subject by at least 35-fold relative to oral administration of the active agent or the salt thereof to a subject. Further provided herein are methods, wherein vaginally administering a composition provided herein reduces systemic concentration of the active agent or the salt thereof in the subject by at least 40-fold relative to oral administration of the active agent or the salt thereof to a subject. Further provided herein are methods, wherein vaginally administering a composition provided herein reduces systemic concentration of the active agent or the salt thereof in the subject by at least 45-fold relative to oral administration of the active agent or the salt thereof to a subject. Further provided herein are methods, wherein vaginally administering a composition provided herein reduces systemic concentration of the active agent or the salt thereof in the subject by at least 50-fold relative to oral administration of the active agent or the salt thereof to a subject.

Provided herein are methods of inducing pelvic pass metabolism of an active agent in a subject, wherein the methods comprise: vaginally administering to a subject a polybioadhesive gel comprising: (a) an active agent or a salt thereof; (b) one or more water-insoluble bioadhesive; (c) at least one oleogel; and (d) at least one aqueous gel, wherein the vaginally administering increases the level of the active agent in the peritoneal tissue relative to the level of the active agent in the serum of the subject, and wherein the vaginally administering maintains a ratio of the level of the active agent in a serum of the subject to the level of the active agent in a peritoneal fluid of the subject that is substantially the same relative to oral administration of an oral pharmaceutical composition comprising a substantially equivalent dose of the active agent or salt thereof, thereby localizing the active agent to the peritoneal tissue and inducing pelvic pass metabolism in the subject. Further provided herein are methods, wherein the vaginally administering is performed once every 24 hours. Further provided herein are methods, wherein the active agent is a synthetic steroid. Further provided herein are methods, the synthetic steroid is danazol or a salt thereof. Further provided herein are methods, wherein the active agent is micronized. Further provided herein are methods, wherein the subject has or is suspected of having infertility. Further provided herein are methods, wherein the subject has or is suspected of having endometriosis. Further provided herein are methods, wherein the vaginally administering reduces the number of endometrial lesions in the subject by at least 10% relative to the number of endometrial lesions in the subject before vaginally administering the polybioadhesive gel. Further provided herein are methods, wherein the vaginally administering reduces the size of one or more endometrial lesions in the subject by at least 10% relative to the size of one or more endometrial lesions prior to vaginally administering the polybioadhesive gel. Further provided herein are methods, wherein the polybioadhesive gel is formulated as a liquid emulsion. Further provided herein are methods, wherein the endometrial disorder is endometriosis, adenomyosis, or a combination thereof. Further provided herein are methods, wherein the polybioadhesive gel does not disrupt ovarian function in the subject. Further provided herein are methods, wherein the polybioadhesive gel reduces pain in the subject by at least 10% relative to pain identified by the subject prior to vaginally administering the polybioadhesive gel. Further provided herein are methods, wherein vaginally administering a composition provided herein reduces systemic concentration of the active agent or the salt thereof in the subject by at least 10-fold relative to oral administration of the active agent or the salt thereof to a subject. Further provided herein are methods, wherein vaginally administering a composition provided herein reduces systemic concentration of the active agent or the salt thereof in the subject by at least 15-fold relative to oral administration of the active agent or the salt thereof to a subject. Further provided herein are methods, wherein vaginally administering a composition provided herein reduces systemic concentration of the active agent or the salt thereof in the subject by at least 20-fold relative to oral administration of the active agent or the salt thereof to a subject. Further provided herein are methods, wherein vaginally administering a composition provided herein reduces systemic concentration of the active agent or the salt thereof in the subject by at least 25-fold relative to oral administration of the active agent or the salt thereof to a subject. Further provided herein are methods, wherein vaginally administering a composition provided herein reduces systemic concentration of the active agent or the salt thereof in the subject by at least 30-fold relative to oral administration of the active agent or the salt thereof to a subject. Further provided herein are methods, wherein vaginally administering a composition provided herein reduces systemic concentration of the active agent or the salt thereof in the subject by at least 35-fold relative to oral administration of the active agent or the salt thereof to a subject. Further provided herein are methods, wherein vaginally administering a composition provided herein reduces systemic concentration of the active agent or the salt thereof in the subject by at least 40-fold relative to oral administration of the active agent or the salt thereof to a subject. Further provided herein are methods, wherein vaginally administering a composition provided herein reduces systemic concentration of the active agent or the salt thereof in the subject by at least 45-fold relative to oral administration of the active agent or the salt thereof to a subject. Further provided herein are methods, wherein vaginally administering a composition provided herein reduces systemic concentration of the active agent or the salt thereof in the subject by at least 50-fold relative to oral administration of the active agent or the salt thereof to a subject. Further provided herein are methods, wherein the wherein upon vaginal administration, the active agent or the salt thereof diffuses into a peritoneal fluid of the subject, reaching further pelvic tissue via transvaginal absorption, thereby localizing the active agent to the peritoneal cavity. Further provided herein are methods, wherein upon vaginal administration, the active agent or the salt thereof diffuses into a peritoneal fluid of the subject, reaching further pelvic tissue via transvaginal absorption, thereby localizing the active agent to the peritoneal cavity.

Provided herein are methods of treating a disease or a condition in a subject, wherein the methods comprise: vaginally administering to the subject a cream provided herein or a composition provided herein, thereby treating the disease or the condition. Further provided herein are methods, wherein the subject has, is suspected of having, or is diagnosed with an endometrial lesion. Further provided herein are methods, wherein the subject has, is suspected of having, or is diagnosed with infertility. Further provided herein are methods, wherein vaginally administering a composition provided herein reduces systemic concentration of the active agent or the salt thereof in the subject by at least 10-fold relative to oral administration of the active agent or the salt thereof to a subject. Further provided herein are methods, wherein vaginally administering a composition provided herein reduces systemic concentration of the active agent or the salt thereof in the subject by at least 15-fold relative to oral administration of the active agent or the salt thereof to a subject. Further provided herein are methods, wherein vaginally administering a composition provided herein reduces systemic concentration of the active agent or the salt thereof in the subject by at least 20-fold relative to oral administration of the active agent or the salt thereof to a subject. Further provided herein are methods, wherein vaginally administering a composition provided herein reduces systemic concentration of the active agent or the salt thereof in the subject by at least 25-fold relative to oral administration of the active agent or the salt thereof to a subject. Further provided herein are methods, wherein vaginally administering a composition provided herein reduces systemic concentration of the active agent or the salt thereof in the subject by at least 30-fold relative to oral administration of the active agent or the salt thereof to a subject. Further provided herein are methods, wherein vaginally administering a composition provided herein reduces systemic concentration of the active agent or the salt thereof in the subject by at least 35-fold relative to oral administration of the active agent or the salt thereof to a subject. Further provided herein are methods, wherein vaginally administering a composition provided herein reduces systemic concentration of the active agent or the salt thereof in the subject by at least 40-fold relative to oral administration of the active agent or the salt thereof to a subject. Further provided herein are methods, wherein vaginally administering a composition provided herein reduces systemic concentration of the active agent or the salt thereof in the subject by at least 45-fold relative to oral administration of the active agent or the salt thereof to a subject. Further provided herein are methods, wherein vaginally administering a composition provided herein reduces systemic concentration of the active agent or the salt thereof in the subject by at least 50-fold relative to oral administration of the active agent or the salt thereof to a subject. Further provided herein are methods, wherein the wherein upon vaginal administration, the active agent or the salt thereof diffuses into a peritoneal fluid of the subject, reaching further pelvic tissue via transvaginal absorption, thereby localizing the active agent to the peritoneal cavity. Further provided herein are methods, wherein the wherein upon vaginal administration, the active agent or the salt thereof diffuses into a peritoneal fluid of the subject, reaching further pelvic tissue via transvaginal absorption, thereby localizing the active agent to the peritoneal cavity.

Provided herein are methods of maintaining ovarian function and reducing pain associated with endometriosis in a subject, wherein the methods comprise: vaginally administering to the subject a cream provided herein or a composition provided herein, thereby maintaining ovarian function and reducing pain associated with endometriosis in a subject. Further provided herein are methods, wherein the subject has, is suspected of having, or is diagnosed with an endometrial lesion. Further provided herein are methods, wherein the subject has, is suspected of having, or is diagnosed with infertility. Further provided herein are methods, wherein vaginally administering a composition provided herein reduces systemic concentration of the active agent or the salt thereof in the subject by at least 10-fold relative to oral administration of the active agent or the salt thereof to a subject. Further provided herein are methods, wherein vaginally administering a composition provided herein reduces systemic concentration of the active agent or the salt thereof in the subject by at least 20-fold relative to oral administration of the active agent or the salt thereof to a subject. Further provided herein are methods, wherein vaginally administering a composition provided herein reduces systemic concentration of the active agent or the salt thereof in the subject by at least 25-fold relative to oral administration of the active agent or the salt thereof to a subject. Further provided herein are methods, wherein vaginally administering a composition provided herein reduces systemic concentration of the active agent or the salt thereof in the subject by at least 30-fold relative to oral administration of the active agent or the salt thereof to a subject. Further provided herein are methods, wherein vaginally administering a composition provided herein reduces systemic concentration of the active agent or the salt thereof in the subject by at least 35-fold relative to oral administration of the active agent or the salt thereof to a subject. Further provided herein are methods, wherein vaginally administering a composition provided herein reduces systemic concentration of the active agent or the salt thereof in the subject by at least 40-fold relative to oral administration of the active agent or the salt thereof to a subject. Further provided herein are methods, wherein vaginally administering a composition provided herein reduces systemic concentration of the active agent or the salt thereof in the subject by at least 45-fold relative to oral administration of the active agent or the salt thereof to a subject. Further provided herein are methods, wherein vaginally administering a composition provided herein reduces systemic concentration of the active agent or the salt thereof in the subject by at least 50-fold relative to oral administration of the active agent or the salt thereof to a subject. Further provided herein are methods, wherein the wherein upon vaginal administration, the active agent or the salt thereof diffuses into a peritoneal fluid of the subject, reaching further pelvic tissue via transvaginal absorption, thereby localizing the active agent to the peritoneal cavity.

### Examples

### Example 1: Formulation of a Composition Comprising a Hormone for Vaginal Delivery

A pharmaceutical composition containing a bioadhesive for delivering a hormone can be prepared for administering vaginally to a subject using the following formulation:

Progesterone Formulation (% w/w):

| | |
|---|---|
| Micronized Progesterone (USP/EP) | 8.0% |
| Carbomer (Carbopol 974P NF) | 1.0% |
| Polycarbophil Noveon AAl (USP) | 1.5% |
| Sorbic Acid (USP/EP) | 0.1% |
| Labrafac Lipophile WL 1349 (USP/EP) | 17.07% |
| EmulFree P Pharma Grade | 2.0% |
| Purified Water (USP/EP) | 68.4% |
| Sodium Hydroxide (USP/EP) QS AD pH 2.8-3.2 | |
| Purified Water (USP/EP) | QS 100% |

The progesterone formulation can be manufactured by dissolving micronized progesterone in an oil phase consisting of LABRAFAC® lipophille WL 1349 and EMULFREE® P. To this suspension, alcohol can be added at a concentration in which the alcohol does not behave as a penetration enhancer. The carbomers will be hydrated in the aqueous phase. After hydration, sorbic acid, polycarbophil, and sodium hydroxide will be added to the aqueous phase. The two phases will be mixed in an appropriate vessel. The provided composition will be stored in a sealed container prior administering vaginally.

In addition to progesterone P (4) any synthetic progestin could be formulated utilizing described formulation.

Estradiol Formulation: (% w/w)

| | |
|---|---|
| Micronized Estradiol (USP/EP) | 0.1% |
| Carbomer (Carbopol 974P NF) | 1.0% |
| Polycarbophil Noveon AA1 (USP) | 1.5% |
| Sorbic Acid (USP/EP) | 0.1% |
| Labrafac Lipophile WL 1349 (USP/EP) | 17.07% |
| EmulFree P Pharma Grade | 2.0% |
| Purified Water (USP/EP) | 76.4% |
| Sodium Hydroxide (USP/EP) QS AD pH 2.8-3.2 | |
| Purified Water (USP/EP) | QS 100% |

Any estrogen could be substituted for estradiol. For example, dienestrol, estriol, or estrone.

Compositions containing a bioadhesive and other active ingredients can be prepared in a similar fashion to the formulations described above Other active ingredients can include other hormones; antineoplastics; receptor agonists or antagonists; steroids; antibiotics; antiviral compounds; antifungal compounds; and anti-inflammatories.

### Example 2: Formulation of a Composition Comprising Danazol for Vaginal Delivery

A pharmaceutical composition containing a bioadhesive for delivering danazol can be prepared for administering vaginally to a subject using the following formulation:

Danazol Formulation: (% w/w)

| | |
|---|---|
| Danazol | 6.67% |
| Carbomer (Carbopol 974P NF) | 1.0% |
| Polycarbophil Noveon AA1 (USP) | 1.5% |
| Methylparaben | 0.25% |
| Labrafac Lipophile WL 1349 (USP/EP) | 15% |
| EmulFree P Pharma Grade | 2.0% |
| Purified Water (USP/EP) | 73.58% |

The danazol formulation can be manufactured by dissolving danazol in an oil phase consisting of LABRAFAC® lipophille WL 1349 and EMULFREE® P. The carbomers will be hydrated in the aqueous phase. After hydration, methylparaben and polycarbophil will be added to the aqueous phase. The two phases will be mixed in an appropriate vessel. The provided composition will be stored in a sealed container prior administering vaginally.

### Example 3: a Comparative, Open-Label, Randomized, Parallel Group Study to Determine the Effects of a Vaginally Applied Formulation Comprising Danazol as a Treatment for Endometriosis

A Comparative, Open-Label, Randomized, Parallel Group Study was performed to determine intraperitoneal fluids, tissue, and serum concentrations of VML-0501 following five days of daily vaginal applications of single dose of VML-0501 (100 mg Danazol), in comparison to five days of Danazol treatment administered as an oral capsules (Danatrol) at a daily dose of 600 mg, in two groups of twelve each consisting of women with suspected or confirmed endometriosis and scheduled for laparoscopy.

Study Objectives: The objectives of the study were: to assess the bioavailability of VML-0501 in comparison to oral Danazol in terms of: concentration of Danazol in serum; and concentration of Danazol in peritoneal fluid. Another objective of the study was to access the concentration of Danazol in endometrial tissue found outside the uterus (lesion tissue). The additional objectives of the study were to access the concentration of Danazol in endometrium tissue. To access the concentration of Danazol in myometrium tissue (only in those patients in which no increased surgical risk was present).

This was a comparative, open-label, randomized parallel group study, investigating the concentrations of Danazol when administered orally and vaginally. Subjects who fulfilled the inclusion and exclusion criteria were randomized in an open-label fashion to be dosed with either VML-0501 (vaginal Danazol) or DANATROL® (oral Danazol) in a 1:1 ratio.

Subjects in the VML-0501 group were administered with 1.5 g of VML-0501 cream (containing 100 mg of Danazol) deeply within the vagina using an internal applicator, followed by 30 minutes of lying on their back in a supine position. Subjects were dosed once daily every morning for 5-7 consecutive days until the date of their laparoscopy.

Subjects in the Danatrol group were dosed with 200 mg of oral Danazol, three times daily (TID) for 5-7 consecutive days until the date of their laparoscopy. Subjects attended a follow up visit 7/8 days after discharge.

Number of Subjects: The actual number of patients enrolled was 30.

Table 2 below includes the Inclusion and Exclusion Criteria for Patients in the trial.

TABLE 2
Inclusion and Exclusion Criteria.

DIAGNOSIS AND MAIN CRITERIA FOR INCLUSION:

| | |
|---|---|
| 1. | Female aged between 18-42 years. |
| 2. | Be able to provide written (personally signed and dated) informed consent before |
| | completingnay study-related procedure, which means any assessment or evaluation |
| | that would not have formed a part of her normal medical care. |
| 3. | Has or is suspected to have endometriosis. |
| 4. | Scheduled to undergo laparoscopy. |
| 5. | According to the local practice the women should sign a specific clause on |
| | avoidingpregnancy based on the use of two effective methods for birth control |
| | (condom and spermicide) following 30 days after the last dose/application. |
| 6. | Be non-pregnant undergoing laparoscopy for confirmed or suspected endometriosis |
| | withinthe first 14 days of her cycle. |
| 7. | Have a Body Mass Index (BMI) of ≤ 32 kg/m² |

Key Exclusion Criteria:
Subjects were not able to be enrolled into the study if they any of the follow criteria applied:

| | |
|---|---|
| 1. | Is pregnant (positive-urine pregnancy test at screening) or lactating. |
| 2. | Has evidence of drug or alcohol abuse. |
| 3. | Have used hormonal replacement therapy or Danazol therapy within the past 8 |
| | weeks beforestudy entry. |
| 4. | Has any of the following: Epileptic Seizure, Migraine Headache, Angina, |
| | Chronic Heart Failure, Obstruction of a Blood Vessel by a Blood Clot, Liver |
| | Problems, Kidney Disease, Pregnancy, Combined High Blood Cholesterol and |
| | Triglyceride Level, Porphyria. |
| 5. | Undiagnosed abnormal genital bleeding. |
| 6. | Androgen dependant tumour. |
| 7. | Is allergic to anabolic androgenic steroid. |
| 8. | Smoker. |

1.5 grams of VML-0501 cream was administered vaginally at a dose of 100 mg of Danazol once daily to each subject. The reference drug in this study was Danatrol containing 200 mg of Danazol per capsule. Danatrol was administered orally three times daily.

DURATION OF TREATMENT: All patients were to receive an allocated treatment of Danazol for 5-7 days or based on the decision of the investigator.

### Criteria for Evaluation:

Efficacy: The primary efficacy endpoint was to measure the concentration of Danazol present in the peritoneal fluid and serum. The secondary endpoint was to measure the concentration of Danazol present in the endometrial tissue (lesion tissue) outside the uterus.

Exploratory endpoints were added mid-study, as part of a protocol amendment, to include the assessment of the concentration of Danazol in endometrium and myometrium tissue (only in those patients in which no increased surgical risk was present).

A bioanalytical method for testing the concentration of Danazol in human serum albumin was developed to evaluate the serum concentration after completing the dose (at steady state), peritoneal fluid and the lesion concentration in the sample of tissue collected at laparoscopy.

Safety: Safety assessments included analysis of AE's, physical examinations, vital signs, blood, and urine tests to investigate the effects of the study drug on clinical tolerability. Adverse events were coded using the WHO Adverse Reaction Terminology. Adverse events were reported by primary body system and preferred term.

### Statistical Methods:

Descriptive statistics were used to summarize all continuous variables (such as clinical demographic characteristics), and included the number of patients, mean, standard deviation, median, first and third quartiles. For categorical variables, per category, the absolute counts and percentages were presented. The primary and secondary endpoints are summarized in tabular summaries and listings. The average value and the 95% confidence internal (CI) or the median value and the interquartile range (depending on the nature of the parameter) was investigated for each endpoint. Paired Student T Test and non-parametric paired Wilcoxon Test were used to compare the serum and peritoneal fluid within each group. Wilcoxon Mann Whitney test was used to compare the ratio between peritoneal fluid and serum between the two groups. Due to the risk of surgery, tissue samples could not be recovered from all patients with detectable Danazol both in serum and peritoneal fluid. Due to the small number of tissue samples collected, data were qualitatively described to report the number of patients with detectable Danazol in tissue samples within each treatment group.

Adverse events and concomitant medications were reported on a per-patient basis. The denominator in percentage calculations represented the number of patients in the population of interest. The selection of subjects to be included in the analysis will be defined as follows: Primary population-Intent to treat-(subjects selected, randomized and have taken at least one dose of the study medication); Secondary population-Per protocol-(subjects who complete the study trial and have taken at least 80% of the study medication); The package SAS® System version 9.4 was used for all analysis. A p-value of <0.05 was considered as statistically significant.

### Example 4: Danazol Composition Clinical Efficacy

Of the thirty patients enrolled in the study, 29 were randomized (16 to the VML-0501 group and 13 to the Danatrol group), with two patients subsequently withdrawing consent, and two patients dropping out due to other reasons. The baseline demographics and characteristics were homogenous amongst the VML-0501 and Danatrol groups. Comparison of serum concentration between the two groups shows that serum concentration was significantly lower in the vaginal group (p<0.001). The comparison of Danazol concentration within each group showed that the concentration was higher in serum (p<0.001) than peritoneal fluid, both when considering the <1 value as 0.5 or as missing.

A similar ratio of Danazol concentration from serum to peritoneal fluid was reported to in both groups, confirming a similar systemic action, and by a different mechanism with vaginally administered VML-00501. The concentration of Danazol was significantly higher in tissue than in the serum samples in the VML-0501 group (n=6, p-0.031), however, did not reach significance in the Danatrol group (n=11, p=0.054).

### Example 5: Safety of the Danazol Composition

There were no Serious Adverse Events (SAEs) or photoallergic reactions observed in either treatment group. There were only three AEs recorded amongst all enrolled subjects (two events were in VML-0501 group and one in the Danatrol group). No significant difference was found between groups with regard to AEs.

VML-0501, a polybioadhesive gel, offers persistence of benefits consistent with direct tissue effects for lower pelvic inflammation. Low serum levels do not affect the hypothalamic-pituitary-axis and can protect ovarian functions, minimize systemic effects, and improve fecundability.

Results from the VML-0501-001 study show significantly lower levels of danazol concentration in the serum with VML-0501 in comparison to oral danazol. The use of low-dose vaginal danazol, which reaches peritoneal fluid and peritoneal cavity, should reduce inflammation with a beneficial effect on the ovarian quality and to immunoregulate the pain signaling in the peritoneal fluid. The concentration of danazol in the tissue biopsies was significantly higher when compared to the serum levels, in the VML-0501 group, suggesting diffusion of the drug into the peritoneal fluid, reaching further pelvic tissue via transvaginal absorption and the counter-current transfer between uterovaginal lymph vessels or veins and arteries keeps the drug in the region.

### Example 6: Methods of Treating Endometriosis

Endometriosis is a condition in women characterized by the presence of endometrial tissue (endometrial glands and stroma) outside the uterine cavity and can commonly be associated with chronic pelvic pain and infertility. An example of these lesions can be peritoneal lesions or deep infiltrating disease. Diagnosis is often delayed due to a lack of consistent diagnostic biomarkers for endometriosis. The efficacy of treatments such as hormone therapy and analgesics used for symptomatic endometriosis is limited due to the high rate of recurrence in many women.

Oral Danazol is a treatment for endometriosis. Danazol is a synthetic steroid, derivative of 17 alpha-ethinyl testosterone, used to suppress ovarian function by enforcing direct inhibitory effects on ovarian steroidogenesis and through the inhibition of gonadotrophins (follicle stimulating hormones (FSH) and luteinizing hormones (LH)). Danazol has mild androgenic effects, however, has no estrogenic or progestational properties. In women with regular menstrual cycles danazol induces the state of pseudomenopause characterized by complete suppression of ovarian function, amenorrhea and hypoestrogenic state. Uterine endometrium and ectopic endometrium undergo atrophy during treatment, resulting in a reduction of endometriosis.

Provided herein are methods of treating endometriosis comprising vaginal administration of 100 mg of danazol in the form of a liquid emulsion. Provided herein is the first comparative study analyzing the different concentrations in serum, peritoneal fluid, and endometrial tissue of vaginally administered 100 mg Danazol, (VML-0501) compared with 600 mg oral danazol capsules (Danatrol®).

Objectives: The primary objective of this study was to investigate and compare the concentration of Danazol present in the serum and the peritoneal fluid of patients in both VML-0501 and Danatrol groups. The secondary objective of this study was to investigate the concentration of Danazol present in the lesion tissue. The exploratory objective of this study was to investigate the concentration of Danazol present in the endometrium and myometrium tissue (if applicable).

### Investigational Plan

### Overall Study Design and Plan

The VML-0501-001 study was a Phase II, parallel arm, open-label, single-center, proof of concept study designed to determine the levels of Danazol concentration in patients with suspected or clinically diagnosed endometriosis, when dosed with oral or vaginal Danazol treatments. The planned enrolment was approximately 24 patients, which was later extended to 34 patients due to the difficulty in recruitment of patient population.

Patients with suspected or clinically diagnosed endometriosis were screened and invited to participate in the study during routine hospital visits. Informed consent was obtained on Day 1, and patients were progressed for a physical examination. Patients height and weight were measured, and vital signs were recorded. Blood and urine samples were taken for baseline analysis, and a pregnancy test was completed for confirmation of eligibility inclusion. All tests were done as part of standard of care during the patient's routine hospital appointment. Patients were informed to attend on Day 4 of their menstrual cycle where they were randomized into either the VML-0501 or Danatrol group.

Physical examination was completed, and vital signs were also recorded during this visit. Patients were provided with a demonstration as to how to complete each dose for the following five to seven days (treatment for some patients was extended to 7 days as a medical decision based on the investigator's discretion) prior to their laparoscopy date. Laparoscopy and sampling was be done before Day 14 of the menstrual cycle (day of laparoscopy varied between patient due to the heaviness of bleeding during menstrual cycle which determined when the start date of the trial treatment was, and electivity of the procedure).

Steady state sampling was completed on either day eight, nine or ten of the menstrual cycle as it would have been achieved within five days of being dosed.

Patients were discharged post-laparoscopy and asked to return for a follow up visit approximately one-week post-discharge. During the follow up visit patients provided a blood and urine sample, and a physical examination was conducted.

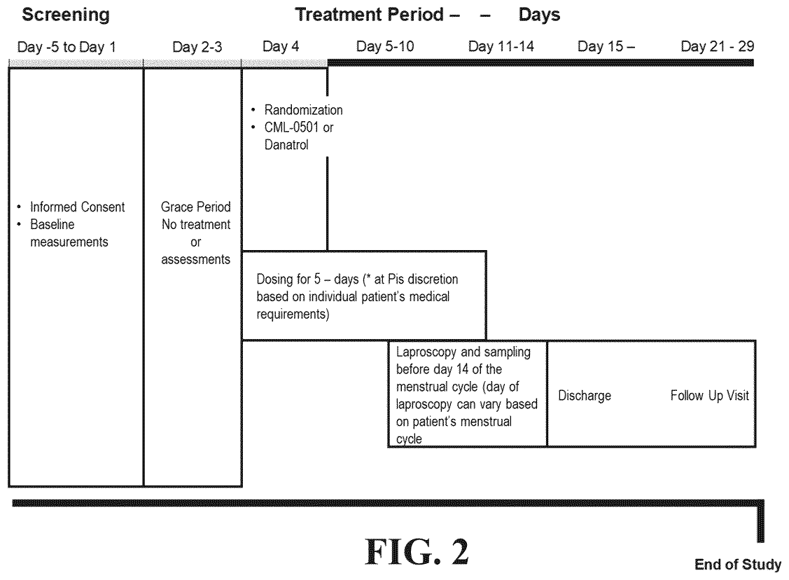

Safety was assessed at every study visit, and also throughout the study based on reporting by the patient in the patient workbook. Any adverse events were monitored closely and the incidence, severity and relationship to the study drug was assessed. FIG. 2 provides a schematic of the study design indicating the timing of study assessments and dosing.

### Study Design Including the Choice of Control Group

Dosing was administered in a parallel open-label manner. Both patients and investigators were unblinded to the treatment allocations. This design was selected to minimize the risk of treating patients with ineffective therapy, and early termination/withdrawal of the study was permitted should the patient or investigator wish so. A total of 34 patients were expected to be randomized, with 17 patients in each arm.

The selected control group for this study was the Danatrol arm. Danatrol, an approved medicine product, is a derivative consisting of 200 mg of Danazol as the active substance per capsule. Danatrol's therapeutic indications include the symptomatic treatment of endometriosis as oral administration. This form of treatment should be started during menstruation alongside an effective, non-hormonal contraceptive method in non-pregnant or lactating women. To reach amenorrhea a stating dose of 600 mg/day of Danatrol should be given, starting on the first day of the menstrual cycle, followed by a maintenance dose as judged a medical professional. Danazol in this form is absorbed from the gastrointestinal tract after which peak-plasma concentrations of 50-80 ng/ml are reach after approximately 2-3 hours [4]. Studies have shown that the bioavailability can be tripled by the intake foods high in fat, however the absolute bioavailability remains unknown. Danazol is very quickly and nearly completely metabolized in the liver. Vaginal application of danazol could potentially bypass the liver to reduce any undesired effects. This choice of control group was made upon the evidence that low dose danazol therapy has been shown to have positive results in endometriosis symptom relief, which was used as a comparator in this study for the same, if not improved, desirable outcome.

The parallel design was used in this study for an equal comparison of danazol concentration between the oral and the vaginal doses.

### Study Population

### Inclusion Criteria

To be eligible for inclusion in the study subjects had to fulfil the following criteria:

Female aged between 18-42 years.

Be able to provide written (personally signed and dated) informed consent before completing nay study-related procedure, which means any assessment or evaluation that would not have formed a part of her normal medical care.

Has or is suspected to have endometriosis.

Scheduled to undergo laparoscopy.

According to the local practice the women should sign a specific clause on avoiding pregnancy based on the use of two effective methods for birth control (condom and spermicide) following 30 days after the last dose/application.

Be non-pregnant undergoing laparoscopy for confirmed or suspected endometriosis within the first 14 days of her cycle.

Have a Body Mass Index (BMI) of ≤32 kg/m2

### Exclusion Criteria:

Subjects were not able to be enrolled into the study if they any of the follow criteria applied:

Is pregnant (positive-urine pregnancy test at screening) or lactating.

Has evidence of drug or alcohol abuse.

Have used hormonal replacement therapy or Danazol therapy within the past 8 weeks before study entry.

Has any of the following: Epileptic Seizure, Migraine Headache, Angina, Chronic Heart Failure, Obstruction of a Blood Vessel by a Blood Clot, Liver Problems, Kidney Disease, Pregnancy, Combined High Blood Cholesterol and Triglyceride Level, Porphyria.

Undiagnosed abnormal genital bleeding.

Androgen dependent tumor.

Is allergic to anabolic androgenic steroid.

Removal of Subjects from Treatment or from the Study

In accordance with the study protocol, withdrawal or premature discontinuation was defined as withdrawal from study procedures after first exposure to study treatment.

Subjects who dropped out before the first administration of either VML-0501 or Danatrol were considered as “screening failures”. Subjects were able to withdraw from the study at their own request, at any time, or based on the investigator's clinical judgment. No disadvantage arose for those who wished to withdraw consent or was taken out of the investigation by the investigator for any reason. Subjects who requested withdraw from the treatment were strongly encouraged to complete appropriate examinations for safety and tolerance assessment.

Subjects withdrawn from the study for medical reasons or at the sponsors request were not followed up in regard to the study (patients attended as per SoC routine for hospital follow ups).

The following categories were used to classify the reason for withdrawal:

Lack of tolerability: unacceptably bad tolerability reported by the subject or observed by the physician; a corresponding AE with causality classified as “likely” should be linked.

Inter-current disease: any disease or condition which occurred during the trial that made further participation impossible; there is, however, no evidence for a causal relationship between the event and the study treatment; there should be a corresponding AE with causality classified as “unlikely” or “not assessable”.

Withdrawal of consent/non-compliance: the subject withdrew consent, or the subject was non-compliant with study procedures; it would be checked whether an adverse event may have caused the withdrawal.

Other reason(s): reason for discontinuation not covered by any of the above criteria, e.g. move of the subject, indication does not apply any more, other reason(s) must be specified.

### Treatments Administered:

VML-0501 or Danatrol was administered to patients who met entry criteria at a dose of either 100 mg of danazol once daily or 200 mg of danazol three times daily respectively, for 5-7 consecutive days over one study period. The length of treatment varied by patient based on the length of their menstrual cycle and the PIs medical decision. The treatment administered was based on which arm of the study the patient was randomized to. Treatment was continued until the day of laparoscopy for each patient.

### VML-0501:

Danazol is a synthetic steroid (chemical compound) with antigonadotropic and anti-estrogenic activities that acts as an anterior pituitary suppressant by inhibiting the pituitary output of gonadotropins. It possesses some androgenic properties. Danazol has been used in the treatment of endometriosis and some benign breast disorders. Danazol is a class II molecule (low solubility high permeation) according to G. Amidon classification (BCS). Log P=4.5 (testosterone=3.22, progesterone=3.82, estradiol=4.01). It was approved by the US Food and Drug Administration as the first drug to specifically treat endometriosis in 1971 and it is marketed widely throughout the world including Italy, where this study was conducted.

Danazol presents as a white to pale yellow crystalline powder. VML-0501 is a cream developed for vaginal application, containing 6.7% (w/w) danazol as the active ingredient. It is formulated with Polycarbophil, sorbic acid (Potassium sorbate), Purified water, Labrafac (Propylene glycol dicaprylocaprate), Carbomer 974P and Emulfree (Propylene Glycol Laurate (and) Ethylcellulose (and) Propylene Glycol Isostearate). It is presented as a 30 g aluminum tube with a vaginal applicator. The quantitative formulation of VML-0501 is provided in the Table 3 below.

TABLE 3
Quantitative Formulation of VML-0501.

| Component | Quantity (% w/v) |
|---|---|
| Micronized Danazol | 6.67 |
| Polycarbophil (Noveon AA-1) | 1.5 |
| Sorbic Acid | 1.0 |
| Emulfree P Pharma | 2.0 |
| Purified Water | 73.73 |
| Labrafac ® lipophile WL 1349 | 15.0 |
| Carbopol 974P | 1.0 |
| qs | 100.00 |

The storage conditions for VML-0501 were below 25° C. and 65+5% relative humidity, which are in accordance with the ICH guidelines. GB Pharma confirmed the IMP was stored in a dry, cool area, away from direct sunlight.

The decision was made to use 100 mg of Danazol in VML-0501 as this is a study requiring a low dose to investigate the effects on serum, peritoneal fluid, and tissue versus that of oral prescriptive doses.

### Comparator: Danatrol

The Danatrol comparator was obtained from a local pharmacy (as this product is commercially available) and was not modified in any way. The supply was sent to MiPharm who labelled and passed on the drug to the hospital pharmacy as per trial protocol. Danatrol capsules of 100 and 200 mg contain as additives corn starch, lactose, talcum and magnesium stearate. The capsule body contains titanium dioxide (E171) and gelatine. Danatrol has a stability of five years and was not stored at temperatures above 25° C. as per manufacturer guidelines. The known plasma half-life of Danatrol varies between 3-6 hours after one single dose.

### Dose Selection and Timing

Doses of oral Danazol at 100-200 mg daily can lead to symptom relief, reduction in heavy menstrual bleeding and reduction in the volume of lesions, as well as possible improvement in fertility in patients with endometriosis. Higher doses of oral Danazol (600-800 mg) daily may be required in more severely affected patients, however the associated side effects have resulted in limited usage of this treatment.

Vaginally administered danazol was tested herein to determine the drug's utility as an effective in the treatment of rectovaginal endometriosis.

Vaginal application allows for direct targeting of lesions in the proximity of the vagina at lower doses than that required for oral administration. The formulation of danazol provided herein results in a higher concentration of the drug distributed to the surrounding areas, and lower levels of the drug circulating. Vaginal application also minimizes the side effects frequently associated with oral intake and does not inhibit ovulation. Vaginal administration also limits systemic propagation of Danazol, therefore minimizing androgenic side effects. The treatment has shown to be well tolerated with patient satisfaction reaching 80%, and the average consumption of pain relief tablets (e.g., naproxen) reduced by 60% in these previously refractory patients. However, its use is contraindicated in pregnancy, even in smaller doses. Vaginal Danazol can be offered as an adjuvant to conventional medical treatments, avoiding contraception issues that may arise with this treatment. It can also be an alternative to performing difficult surgery in patients with deep infiltrating endometriosis of the rectovaginal septum.

For the selected population of suspected and clinically diagnosed endometriosis patients, it was decided that the daily suitable dose of vaginal danazol in the IMP would be 100 mg (⅙ of total comparator dose), and Danatrol would be 600 mg as a fair comparison. The study dosing regime is summarized in Table 4 below.

TABLE 4
Comparison of VML-0501 and Danatrol Administration.

| | VML-0501 (100 mg) | Danatrol (600 mg) |
|---|---|---|
| Delivery | Vaginal | Oral |
| Method | Cream | Capsule |
| Single Dose | 100 mg of Danazol | 200 mg of Danazol |
| Frequency | Once daily | Three times daily (TID) |
| Duration | 5-7 days | 5-7 days |
| Special Requirements | Patient laid in supine position | Patient to consume |
| | for 30 minutes after | capsule with minimum of |
| | administration of VML-0501 | 250 mL of water |

### Blinding

This was a parallel-arm, open label, proof of concept study that was not blinded. The aim of this study was to obtain further information about the safety and mechanism of action of VML-0501 using a new delivery method differing form the conventional oral therapy.

### Prior and Concomitant Medication

All concomitant medications (con meds) were reported in on an on-going basis from start of treatment through to the final follow up visit as per protocol requirements.

The use of local or systemic Hormonal Replacement Therapy (HRT), including estrogens, within the preceding weeks prior to treatment were not permitted. The uses of local and systemic estrogen phototherapy were also not permitted. Participants were required not to use lubricating agents during sexual intercourse throughout the duration of the study.

Patients could continue medication for any underlying diseases which were already present at baseline (visit 1), and which did not make them ineligible for the study. Prior therapies were only reported if they were significant and concomitant during the time of enrolment.

### Treatment Compliance

Subjects received the treatments at the investigational sites and self-administered at home up until the day of their laparoscopy appointment. Study treatment compliance was assessed through the monitors' review of the dose administration, AE's reported, and the drug accountability upon receiving the assigned IMP/comparator (empty/partially/unused filled tubes/boxes) back from the subjects at the end of the study.

Monitors confirmed that the receipt of all drug supplies at the site and the batch numbers correlated accordingly in line with the drug accountability records.

The full listing of study procedures is provided below in Table 5.

TABLE 5
Study procedures.

| Menstrual day | | | | | | | | | | | Follow-up |
|---|---|---|---|---|---|---|---|---|---|---|---|
| Day 1 | Day 2 | Day 3 | Day4 | Day 5 | Day 6 | Day 7 | Day 8 | Day 9 | Day 10 | Discharge | visit |

| | | | | | | | | | | |
|---|---|---|---|---|---|---|---|---|---|---|
| Study visit day | | 1 | 2 | 3 | 4 | 5 | 6 | 7 | | |
| Screening | X | | | | | | | | | |
| Informed consent | X | | | | | | | | | |
| Physical examination/US | X | X | | | | | | | X | X |
| Ht and Wt. | X | X | | | | | | | | |
| BP and HR | X | X | | | | | | | X | |
| Pregnancy test | X | | | | | | | | | |
| Blood tests | X | | | | | | | | | X |
| Urine tests | X | | | | | | | | | X |
| Randomisation | | X | | | | | | | | |
| VML-0501 or Oral | | X | X | X | X | X | X | X | | |
| Danazol(Danatrol)² | | | | | | | | | | |
| Laparoscopy including | | | | | | X | X | X | | |
| peritoneal and lesion | | | | | | | | | | |
| sampling² | | | | | | | | | | |
| Steady State Blood Draw³ | | | | | | X | X | X | | |
| Adverse event | | → | → | → | → | → | → | → | → | → |

¹ VML-0501 to be applied at 8.00am daily until the laparoscopy oral Danazol (Danatrol) 200 mg 3 times daily to be taken until the laparoscopy.
²Laparoscopy and sampling could be done on either day 8/9/10 of menstrual cycle
³Steady state sampling could be done on either day 8/9/10 of menstrual cycle as the it will be achieved in 5 days of dosing.
⁴ Follow up visit can be scheduled after 7/8 days of discharge.

A number of assessments were performed during the Screening period to determine patient eligibility. Local pathological reviews and diagnosis were used to determine if a patient met the requirements of having suspected or clinically diagnosed endometriosis as per the inclusion criteria. Trained medical personal and the investigator were responsible for the screening of determination of eligibility of patients.

In addition, a urine pregnancy test for women of childbearing potential was performed within three days prior to the first dose.

### Medical History and Physical Examinations

A medical history evaluation and a physical examination was completed as part of standard care within 4 days prior to the projected start of Danazol administration, and the following data were recorded: date of birth, age, height, weight, BMI, blood pressure, heart rate, details of any ongoing medical conditions and medications taken prior to Danazol administration (relevance to the on-going conditions and medication were assessed by the investigator for danazol suitability).

### Efficacy Measurements

Efficacy was assessed by the evaluation of concentration levels of Danazol in the serum, peritoneal fluid and tissue. Hematology and serum chemistry, coagulation, and urinalysis assessments were performed at Baseline (within 3 days prior to the projected start of Danazol administration) and during the End of Study Follow-up Visit. Therefore, a comparison of both before and after danazol administration could be made.

The primary efficacy endpoint analysis was pre-defined to be based on the comparison of the concentrations of danazol in the afore mentioned parameters within and between patients in both arms of the study (VML-0501 vs. Danatrol).

### Adverse Events

Symptoms, AEs and intercurrent illnesses were monitored throughout the study, and recorded during every visit. AE's were coded according to the event(s) and disease history in accordance with the Medical Dictionary for Regulatory Activities (MedDRA™) coding system.

### Laboratory Measurements

Laboratory assessments were performed at Baseline and End of Study Follow-up Visit as part of standard of care. A blood sample was collected on either Day 8, 9 or 10 of the study for drug steady state analysis, based on the patient's menstrual cycle and the investigators decision. Oral Danazol is known to reach steady state within seven days from the day of dosing.

### Vital Signs

Blood pressure and heart rate measurements were performed at Baseline, Day 1 of dosing, and during discharge as part of standard of care for all patients.

### Efficacy Variables

The primary efficacy endpoint to determine the concentration of danazol was evaluated using a validated assay following EMA and FDA criteria, for the bioanalytical quantification for danazol in human serum albumin. Measurements were compared between serum and peritoneal fluid as well as within the groups as an individual comparison between the VML-0501 and Danatrol groups. The secondary efficacy endpoint was to measure the concentration of danazol in the endometrial tissue which was found outside the uterus (lesion tissue), and in cases whereby patients had no increased surgical risk the concentration of Danazol would be explored in the endometrium and myometrium tissue as an exploratory endpoint.

### Safety Variables

The safety variables included the incidence and severity of adverse events and serious adverse events (SAEs), concomitant medication, premature withdrawals, physical examination parameters, vital signs, menstruation dates and routine laboratory test measurements (blood and urine tests as per SoC).

### Statistical and Analytical Plans

VML-0501 was a Phase II, parallel arm, open-label, study. The primary objective and endpoint of the study was to determine the concentration of danazol present in the serum and the peritoneal fluid of patients with suspected or clinically diagnosed endometriosis. Data sets were produced analysis reviews for a total of 30 patients.

The clinical demographics characteristics of the patients were summarized in listings and tables as mean, standard deviation (sd), median and interquartile range (Q1-Q3) for all continuous variables. For categorical variables the data was listed as absolute counts (n) and percentages. These were compared separately for the VML-0501 and Danatrol groups.

The average value and the 95% confidence interval or the median value and the interquartile range (depending on the nature of the parameter) was calculated for each primary and secondary endpoint. The T Student Test and Mann Whitney's Test was used to compare the main endpoints between the VML-0501 and Danatrol groups.

Safety analysis was reported by listings of adverse events and concomitant medication, reported on a per subject basis. The SAS® System package, Version 9.4, was used for all analysis. A p-value of <0.05 was considered to be statistically significant.

### Determination of Sample Size

As this was a proof of concept study the sample size of 34 patients was selected based on practical rather than statistical considerations. VML-0501 is at an early stage of clinical drug development. This was a POC study in order to demonstrate that vaginal delivery of 100 mg of Danazol at ⅙th of the oral dose could potentially reach therapeutic pelvic concentrations (pelvic tissues and peritoneal fluid) similar to 600 mg of oral treatment (Oral danazol 200 mg capsule TID).

The selection of subjects who will be involved in the analysis is as follows:

Safety Set (SAF): This included all the subjects that enrolled in the study and received at least one dose of the investigational drug.

Primary population: Intent to treat (ITT). These are subjects who have been selected, randomized and took at least one dose of the study medication.

Secondary population: Per protocol (PP). These are subjects who have completed the study and took at least 80% of the study medication.

### Efficacy Evaluation

### Data Sets Analyzed

The Safety Set (N=30) included all the subjects that enrolled in the study and received at least one dose of the investigational drug.

The Intent to treat (ITT) (N=29) population included all subjects who have been selected, randomized and took at least one dose of the study medication.

The Per protocol (PP) (N=26) population included all subjects who have completed the study and took at least 80% of the study medication.

### Demographics

Table 6 below summarizes the patient demographics for the ITT population. All patients were female between the ages of 25 and 47 years old. The median age was 35.0 for the full ITT population, with an average BMI of 21.8.

TABLE 6
Patient Demographics.

| | | | |
|---|---|---|---|
| Summary | | | |
| statistics | VML-0501 (100 mg) | Danatrol (600 mg) | Total |

Age (years)

| N | 16 | (100.0%) | 13 | (100.0%) | 29 | (100.0%) |
|---|---|---|---|---|---|---|
| Mean ± SD | 35.1 ± 4.9 | | 35.1 ± 4.2 | | 35.1 ± 4.5 | |
| Median (IQR) | 35.0 | (33-36) | 36.0 | (33-37) | 35.0 | (33-36) |

| | | | |
|---|---|---|---|
| Min-Max | 28-47 | 25-42 | 25-47 |

Height (cm)

| N | 16 | (100.0%) | 13 | (100.0%) | 29 | (100.0%) |
|---|---|---|---|---|---|---|
| Mean ± SD | 163.1 ± 6.7 | | 165.4 ± 7.7 | | 164.1 ± 7.2 | |
| Median (IQR) | 164.5 | (157-169) | 165.0 | (161-170) | 165.0 | (160-169) |

| | | | |
|---|---|---|---|
| Min-Max | 150-172 | 153-180 | 150-180 |

Weight (kg)

| N | 16 | (100.0%) | 13 | (100.0%) | 29 | (100.0%) |
|---|---|---|---|---|---|---|
| Mean ± SD | 60.8 ± 9.6 | | 55.9 ± 6.8 | | 58.6 ± 8.7 | |
| Median (IQR) | 60.5 | (54-65) | 56.0 | (52-58) | 58.0 | (53-62) |

| | | | |
|---|---|---|---|
| Min-Max | 45-82 | 43-72 | 43-82 |

Body Mass Index (BMI)

| N | 16 | (100.0%) | 13 | (100.0%) | 29 | (100.0%) |
|---|---|---|---|---|---|---|
| Mean ± SD | 22.8 ± 2.9 | | 20.5 ± 2.4 | | 21.8 ± 2.9 | |
| Median (IQR) | 22.1 | (21-25) | 19.8 | (19-20) | 20.8 | (20-23) |

| | | | |
|---|---|---|---|
| Min -Max | 19-28 | 18-28 | 18-28 |

Medical History: Most patients presented with a normal general appearance of all body systems. Nopatients experienced any further changes to their baselines measurements upon the followup visit physical examination.

Compliance: All patients had a compliance rate of at least 80% to protocol and dosage requirements.

### Example 7: Efficacy of VML-0501

### Analysis of Efficacy

The primary and secondary endpoints were to compare the concentration of danazol between the serum, peritoneal fluid, and tissue. The data from the ITT population was analyzed.

### Primary Endpoint

Table 7 below shows the concentration of danazol in the serum and the peritoneal fluid for the ITT population of the VML-0501 and Danatrol groups. The concentration of danazol was presented using two different approaches where a value was <1 (i.e. below the limit of detection) was present: The value was either assigned as 0.5 or the value was treated as missing data.

TABLE 7
Concentration of Danazol in serum and in peritoneal fluid in the ITTpopulation

| | | |
|---|---|---|
| Summary | | |
| statistics | VML-0501 (100 mg) | Danatrol (600 mg) |

If <1 is set as 0.5

| Concentration | N | 16 | (100.0%) | 13 | (100.0%) |
|---|---|---|---|---|---|
| in Serum | Mean + SD | 3.7 ± 1.5 | | 155.7 ± 46.6 | |
| (ng/ml) | Median (IQR) | 3.7 | (3.1-4.4) | 155.7 | (113.0-189.0) |
| | Min-Max | 1-7 | | 91-252 | |
| Concentration | N | 16 | (100.0%) | 13 | (100.0%) |
| in peritoneal | Mean ± SD | 1.4 ± 1.5 | | 77.5 ± 51.2 | |
| fluid (ng/ml) | Median (IQR) | 1.1 | (0.5-1.6) | 77.5 | (19.0-131.0) |

| | | |
|---|---|---|
| Min-Max | 1-7 | 2-138 |

If <1 is set as missing

| Concentration | N | 16 | (100.0%) | 13 | (100.0%) |
|---|---|---|---|---|---|
| in peritoneal | Mean = SD | 2.2 ± 1.3 | | 77.5 ± 51.2 | |
| fluid (ng/ml) | Median (IQR) | 2.2 | (1.6- 2.2) | 77.5 | (19.0-131.0) |

| | | |
|---|---|---|
| Min-Max | 1-7 | 2-138 |

Comparison of the concentration of Danazol in the serum samples for both VML-0501 and Danatrol groups showed that the concentration in serum samples was significantly lower in the VML-0501 (P<0.001). However, when compared within each treatment group, the concentration in serum samples was shown to be higher (p>0.001) than in the peritoneal fluid, both considering any values less than 1 as 0.5 or as missing.

The ratio between the concentration of Danazol in serum and peritoneal fluid is not significantly different between the VML-0501 and Danatrol groups (p=0.417 if <1 is set as 0.5; and p=0.369 if <1 is set as missing).

TABLE 8
Ratio of Danazol concentration between serum
and peritoneal fluid in the ITT population.

| | | | | | |
|---|---|---|---|---|---|
| Summary | | | | | |
| statistics | VML-0501 | (100 mg) | Danatrol | (600 mg) | P value |

If <1 is set as 0.5

| Serum and | N | 16 | (100.0%) | 13 | (100.0%) | 0.417 |
|---|---|---|---|---|---|---|
| peritoneal | Mean ± SD | 0.4 ± 0.3 | | 0.5 ± 0.3 | | |
| fluid (ratio) | Median (IQR) | 0.4 | (0.1-0.6) | 0.5 | (0.2-0.6) | |

| | | |
|---|---|---|
| Min-Max | 0-1 | 0-1 |

If <1 is set as missing

| Serum and | N | 16 | (100.0%) | 13 | (100.0%) | 0.369 |
|---|---|---|---|---|---|---|
| Peritoneal | Mean ± SD | 0.6 ± 0.3 | | 0.5 ± 0.3 | | |
| fluid (ratio) | Median (IQR) | 0.6 | (0.4- 0.7) | 0.5 | (0.2-0.6) | |

| | | |
|---|---|---|
| Min-Max | 0-1 | 0-1 |

The comparison of serum concentration between the VML-0501 and Danatrol groups concluded that the mean serum concentrations were significantly lower in the VML-0501 group by a mean of approximately 40-fold (p<0.001). The ratio of danazol concentration between the serum and peritoneal fluid is not significantly different between the VML-0501 and Danatrol groups (p=9.640 if <1 is set as 0.5, and p=0.350 if <1 is set as missing).

TABLE 9
Concentration of Danazol in serum and in peritoneal fluid in the Population

| | | |
|---|---|---|
| Summary | | |
| statistics | VML-0501 (100 mg) | Danatrol (600 mg) |

If <1 is set as 0.5

| Concentration in | N | 15 | (100.0%) | 11 | (100.0%) |
|---|---|---|---|---|---|
| Serum (ng/ml) | Mean ± SD | 3.7 ± 1.5 | | 155.7 ± 51.0 | |
| | Median (IQR) | 3.6 | (3.0-4.4) | 141.0 | (111.0-198.0) |
| | Min-Max | 1-7 | | 91-252 | |
| Concentration in | N | 15 | (100.0%) | 11 | (100.0%) |
| peritoneal fluid | Mean ± SD | 1.4 ± 1.6 | | 77.5 ± 56.0 | |
| (ng/ml) | Median (IQR) | 1.0 | (0.5-1.6) | 88.0 | (16.0-133.0) |

| | | |
|---|---|---|
| Min-Max | 1-7 | 2-138 |

If <1 is set as missing

| Concentration in | N | 15 | (100.0%) | 11 | (100.0%) |
|---|---|---|---|---|---|
| peritoneal fluid | Mean ± SD | 2.2 ± 1.3 | | 77.5 ± 56.0 | |
| (ng/ml) | Median (IQR) | 2.2 | (1.6- 2.2) | 88.0 | (16.0-133.0) |

| | | |
|---|---|---|
| Min-Max | 1-7 | 2-138 |

TABLE 10
Ratio of Danazol concentration between the serum
and peritoneal fluid inthe PP population

| | | | |
|---|---|---|---|
| Summary | VML- | Danatrol | |
| statistics | 0501 (100 mg) | (600 mg) | P value |

If <1 is set as 0.5

| Serum and Peritoneal | N | 15 | (100.0%) | 11 | (100.0%) | 0.640 |
|---|---|---|---|---|---|---|
| Fluid (ratio) | Mean ± SD | 0.4 ± 0.4 | | 0.5 ± 0.3 | | |
| | Median (IQR) | 0.4 | (0.1-0.7) | 0.5 | (0.1-0.7) | |

| | | |
|---|---|---|
| Min-Max | 0-1 | 0-1 |

If <1 is set as missing

| Serum and Peritoneal | N | 15 | (100.0%) | 11 | (100.0%) | 0.350 |
|---|---|---|---|---|---|---|
| Fluid (ratio) | Mean ± SD | 0.6 ± 0.3 | | 0.5 ± 0.3 | | |
| | Median (IQR) | 0.6 | (0.4-0.8) | 0.5 | (0.1-0.7) | |

| | | |
|---|---|---|
| Min-Max | 0-1 | 0-1 |

The secondary endpoint was to investigate the concentration of Danazol measured in endometrial tissue. Due to the risk of surgery, tissue samples could not be collected from all 26 patients who had detectable levels of danazol. Insufficient sample mass was collected from 4 of the 15 patients (27%) in the VML-0501 group.

Detectable values of Danazol were available for 6 of the 11 patients (55%) in the VML-0501 group (range 1.3-5) and for 11 (100%) in the Danatrol group (range 3.2-541). The concentration of Danazol was significantly higher in tissue than in the serum samples in the VML-0501 group (n=6, p-0.031), however, did not reach significance in the Danatrol group (n=11, p=0.054).

In summary, it is evident that there are lower levels of Danazol in the serum when administered vaginally using the VML-0501 cream in comparison to orally administered Danazol.

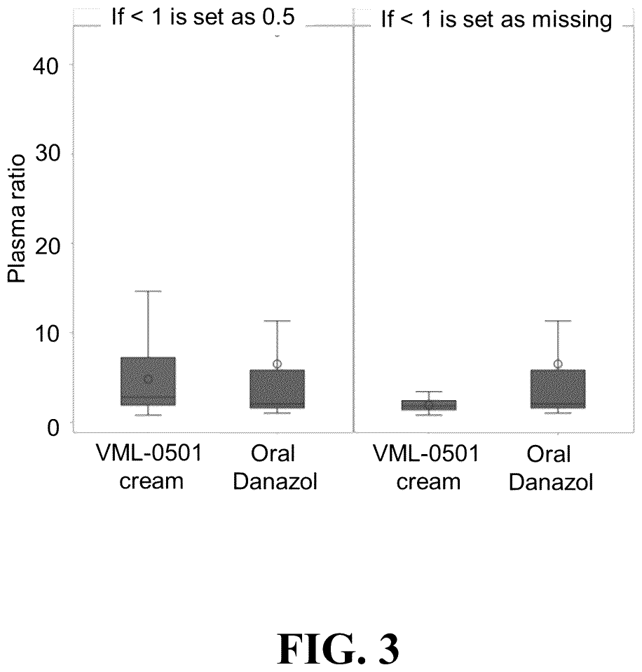

FIG. 3 shows a graph of the ratio of Danazol concentration between Serum and Peritoneal fluid in the (ITT) population.

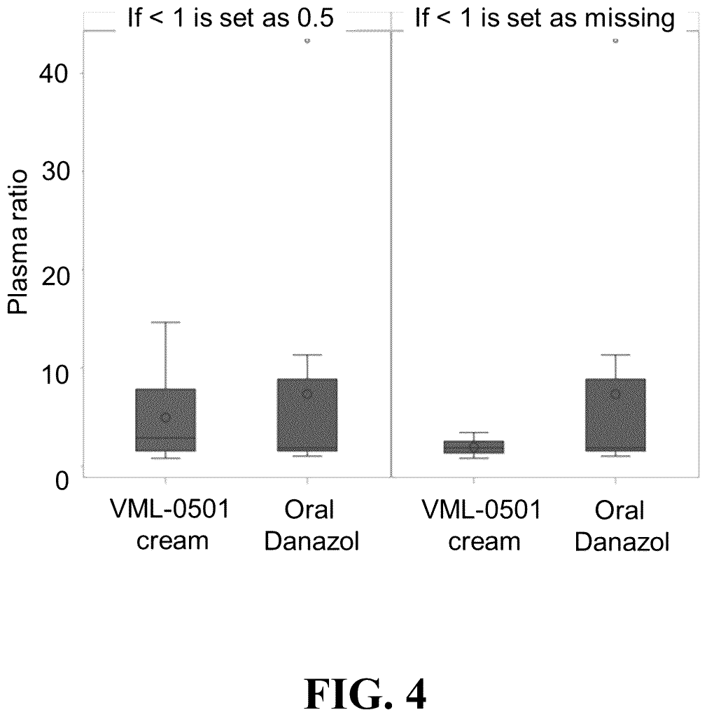

FIG. 4 shows a graph of the ratio of Danazol concentration between Serum and Peritoneal fluid in the PP population.

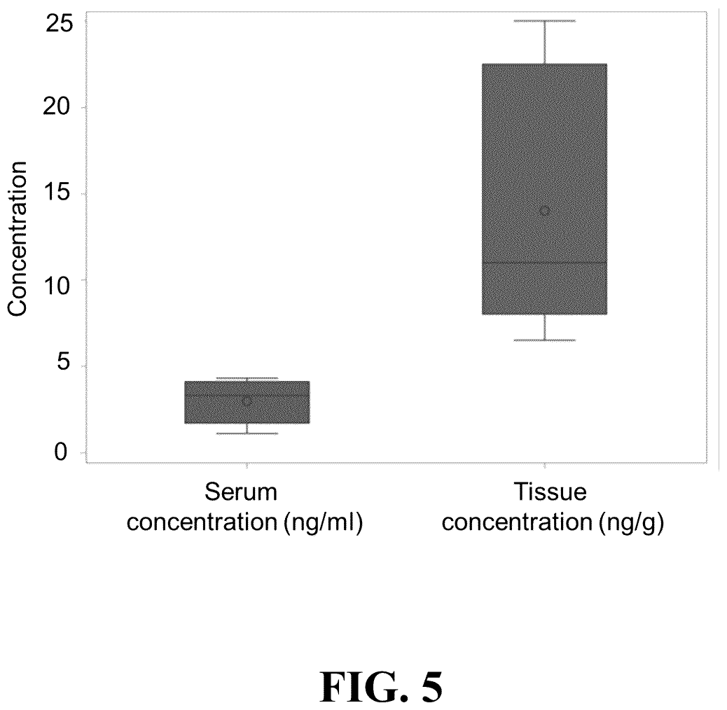

FIG. 5 shows a graph of Danazol in Serum and tissue concentration in VML-0501 dosed group.

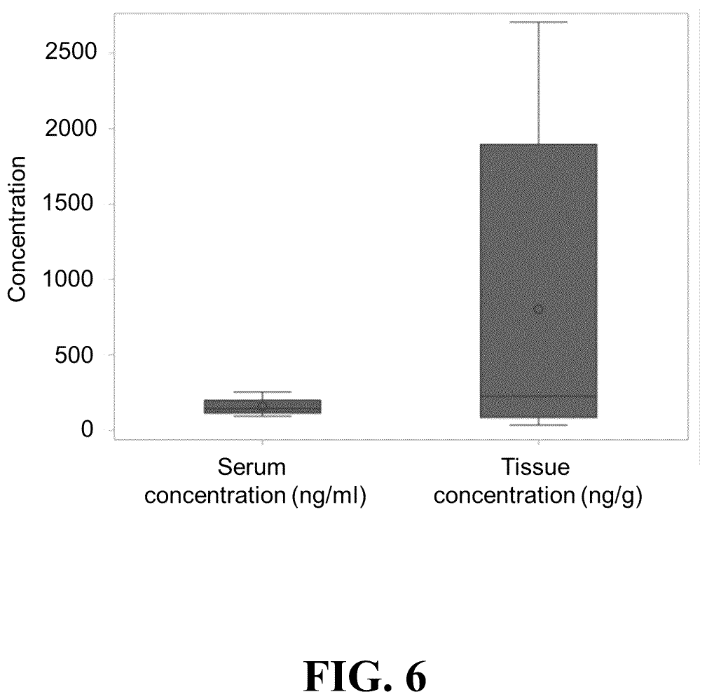

FIG. 6 shows a graph of the concentration of danazol in Serum and Tissue in Oral Danazol group.

### Example 8: Safety Evaluation of VML-0501

All safety analyses were performed on the full analysis dataset (Safety set), which included all patients which included all the subjects that enrolled in the study and received at least one dose of VML-0501.

An overall summary of the extent of danazol exposure is provided in the Table below for the Safety Set.

TABLE 11
Extent of Exposure.

| Patient Number | Treatment Group | Days of dosing |
|---|---|---|
| 01-001 | VML-0501 (100 mg) | 5 |
| 01-002_1 | VML-0501 (100 mg) | 4 |
| 01-002_2 | VML-0501 (100 mg) | 5 |
| 01-003_1 | VML-0501 (100 mg) | 5 |
| 01-004 | Danatrol (600 mg) | 6 |
| 01-005 | Danatrol (600 mg) | 6 |
| 01-006 | Danatrol (600 mg) | 6 |
| 01-007 | VML-0501 (100 mg) | 5 |
| 01-008 | VML-0501 (100 mg) | 5 |
| 01-009 | VML-0501 (100 mg) | 5 |
| 01-010 | Danatrol (600 mg) | 6 |
| 01-011_1 | Danatrol (600 mg) | 5 |
| 01-011_2 | Danatrol (600 mg) | 6 |
| 01-012 | Danatrol (600 mg) | 5 |
| 01-013 | VML-0501 (100 mg) | 4 |
| 01-014 | VML-0501 (100 mg) | 6 |
| 01-015 | VML-0501 (100 mg) | 6 |
| 01-016 | Danatrol (600 mg) | 6 |
| 01-017 | Danatrol (600 mg) | 6 |
| 01-018 | Danatrol (600 mg) | 6 |
| 01-019 | VML-0501 (100 mg) | 6 |
| 01-020 | VML-0501 (100 mg | 7 |
| 01-021 | VML-0501 (100 mg) | 6 |
| 01-022 | Danatrol (600 mg) | 0 |
| 01-023 | Danatrol (600 mg) | 5 |
| 01-024 | Danatrol (600 mg) | 5 |
| 01-025 | VML-0501 (100 mg | 5 |
| 01-026 | VML-0501 (100 mg) | 5 |
| 01-027 | VML-0501 (100 mg) | 5 |

### Brief Summary of Adverse Events:

Adverse Events regardless of causality were collected for all patients from the time of first dose and through to the day of final follow-up visit. An overall summary of the AEs observed in this study are presented in Table 13. An adverse event was defined as any untoward medical occurrence in a patient administered with an investigational product, which does not necessarily have a causal relationship with the treatment itself.

The proportion of subjects experiencing at least one event with 95% Clopper-Pearson Cis was calculated for the total AE and distinguished by treatment group. A total of three out of the 30 subjects enrolled experienced adverse events in this study.

Each of the three subjects reported one AE (10.3%, CI 95%, 0.02-0.27), with two events reported from the VML-0501 group (12.5%, CI 95%, 0.02-0.38), and one event reported from the Danatrol group (7.69%, CI 95%, 0.00-0.36).

TABLE 12
Adverse events from all treatment groups of the study.

| Treatment | n | % | CI 95% |
|---|---|---|---|
| VML-0501 (100 mg) | 2 | 12.50 | 0.02-0.38 |
| Danatrol (600 mg) | 1 | 7.69 | 0.00-0.36 |
| Total | 3 | 10.34 | 0.02-0.27 |

There were no Serious Adverse Events reported. In conclusion there were no safety related concerns within in either treatment group. There were also no statistically significant differences between the two groups in terms of incidence of adverse events. No deaths, or SAEs were reported as a result of any of the treatments.

### Example 9: Comparison of Vaginal and Oral Delivery of Danazol

Oral Danazol decreases estrogen production by the ovaries and endometrial lesions easing the symptoms of endometriosis but causes unpleasant androgenic side effects. Danazol has a high binding affinity to progesterone and androgen receptors which have a direct effect on endometrial lesions resulting in induced apoptosis (cellular programmed death) This can occur as a result of secondary messaging at the cellular level and interruption of DNA synthesis. Aside from pain symptoms associated commonly with endometriosis, up to 50% of women who present with infertility have symptoms related to endometriosis. In fact, biochemically, endometriosis can have a detrimental effect on oocyte quality or on endometrial receptivity.

Current therapies for endometriosis focus on systemic ovarian suppression, which starves the endometriotic lesions of their ability to grow. This therapy induces in the woman a temporary chemical menopausal state resulting in a lack of ovulation, loss of bone loss, mood swings, hot flushes, and other potential debilitating consequences. This results in the need for “add-back” therapy to offset these unintended effects. Additional molecular actions for Danazol include, for example improvement of the activity of immune cells (e.g. natural killer cells) and increased Fas-induced apoptosis for endometrial cells, and anti-inflammatory effects within the pelvic cavity. Oral Danazol is metabolized in the liver by enzymes such as CYP3A4 and can also elevate other liver enzymes. Patients receiving a daily dose, 400 mg or more, of danazol capsules can have elevated serum enzymes and/or jaundice.

As provided herein, bypassing the liver via targeted vaginal administration of danazol can reduce the undesired side effects of hepatic dysfunction and hepatic toxicity observed with oral danazol. Lower doses of vaginally administered Danazol have better side-effect profiles avoiding most of the above-mentioned adverse effects due to bypassing the first liver pass.

VML-0501 utilizes targeted vaginal delivery technology to facilitate the uptake of Danazol from its application site at the cervix to the peritoneal cavity. VML-0501 can target the pelvic organs with drug whilst limiting systemic circulation. Without being bound by a particular theory, this effect can occur due to a counter-current mechanism where the arteries and veins, which are situated in close proximity to one another, flow in opposite directions, thereby preventing significant amounts of drug from being absorbed systemically. Furthermore, VML-0501 is absorbed in the peritoneal cavity through transvaginal epithelial absorption. Danazol reaches the vaginal arteries, with minimum leakage in the systemic circulation and is drawn back into the pelvic cavity, peritoneal organs, and peritoneal fluid. It is also transported from a network of capillaries (rete mirabilis), with a counter-current transfer between uterovaginal lymph vessels or veins and arteries aiming to maintain high concentration locally, and allows danazol binding to endometrial tissue and ovarian tissue. This goes beyond first uterine pass metabolism. Therefore, the compositions and methods provided herein generate a new pharmacokinetic profile for a drug, referred to herein as pelvic pass metabolism.

VML-0501 causes atrophy of endometrial lesions without the need to inhibit the ovarian-pituitary axis. This is achieved without systemic levels of the danazol drug on which other therapies rely and thus preventing the debilitating side effects. In normally cycling women oral administration of danazol induces the state of pseudomenopause, characterized by complete suppression of ovarian function, amenorrhea and hypoestrogenic state. Uterine endometrium and ectopic endometrium undergo atrophy during treatment, resulting in a regression and disappearance of endometriosis.

Results from the VML-0501-001 study show significantly lower levels of danazol concentration in the serum in comparison to oral danazol. Serum concentrations (16/16) in those dosed vaginally showed a significantly lower level of danazol (Mann-Whitney p=0.0001233) when compared to the group dosed orally. The drug was detected in peritoneal fluids and quantified in most samples (12/16). Analyses of the ratio between measured serum concentrations and measured peritoneal fluid concentrations, showed no statistical difference between women receiving VML-0501 and oral Danatrol (Mann-Whitney p=0.2773).

The use of low-dose vaginal danazol, which reaches peritoneal fluid and the peritoneal cavity, reduces inflammation with a beneficial effect on the ovarian quality and can immunoregulate the pain signaling in the peritoneal fluid.

Where pelvic biopsies (tissue found outside the uterus including ovaries, endometrioma capsules, ovarian fossa, broad ligament, utero sacral ligament and peritoneum) were obtained (12/16) danazol was detected in all samples. The concentration of danazol in the tissue biopsies was significantly higher (p=0.031), when compared to the serum levels, in the vaginal danazol group. The same was not observed when the concentration of danazol in the pelvic tissues were analyzed in the oral group.

A similar ratio of Danazol concentration from serum to peritoneal fluid was reported to in both groups, confirming a similar systemic action, and very much a different mechanism with vaginally administered VML-00501. The limited leakage of danazol after the vaginal administration of VML-0501 further protects the patient from unwanted systemic side effects.

The relative higher concentrations achieved in the pelvic tissues (through biopsies) suggests diffusion of the drug into the peritoneal fluid and reach further pelvic tissue via transvaginal absorption and the counter-current transfer between uterovaginal lymph vessels or veins and arteries keeps the drug in the region.

Vaginal administration of danazol presented with zero treatment related adverse events or side effects. No serious adverse events were recorded in either arm of the study. This indicates that the vaginal route of administration using the VML-0501 cream could target the endometriotic lesions to provide symptomatic relief without the undesired side effects seen with oral danazol administration.

The low serum concentrations of vaginally administered danazol was very surprising for a couple reasons. First, the pelvic cavity is highly vascularized. Thus, vaginal administration of VML-0501 would be expected to produce comparable serum concentrations of danazol when compared to oral administration of Danatrol®. That VML-0501 would have been expected to produce similar danazol serum concentrations to orally administered Danatrol®, was also expected because vaginal VML-0501 administration was expected to avoid first pass metabolism in the liver.

Surprisingly, VML-0501 had a markedly reduced danazol serum concentration when compared to orally administered Danatrol®. Markedly lower serum concentrations give rise to fewer adverse effects.

Orally administered danazol can cause many unwanted side effects including cardiac and hepatic toxicity. Specifically, orally administered Danatrol® is associated with undesirable effects such as acne, hair loss, fluid retention, weight gain, increased insulin resistance in diabetic patients, nervous system disorders (e.g., anxiety and depression), nausea, cramping, among others.

In contrast to oral administration, vaginally administered VML-0501 presented with zero treatment related adverse events or side effects. No serious adverse events were recorded in either arm of the study. This result indicates that the vaginal route of administration using the VML-0501 emulsion targets endometriotic lesions to provide symptomatic relief without the undesired side effects seen with oral danazol administration. Thus, the unexpected result is also a superior result.

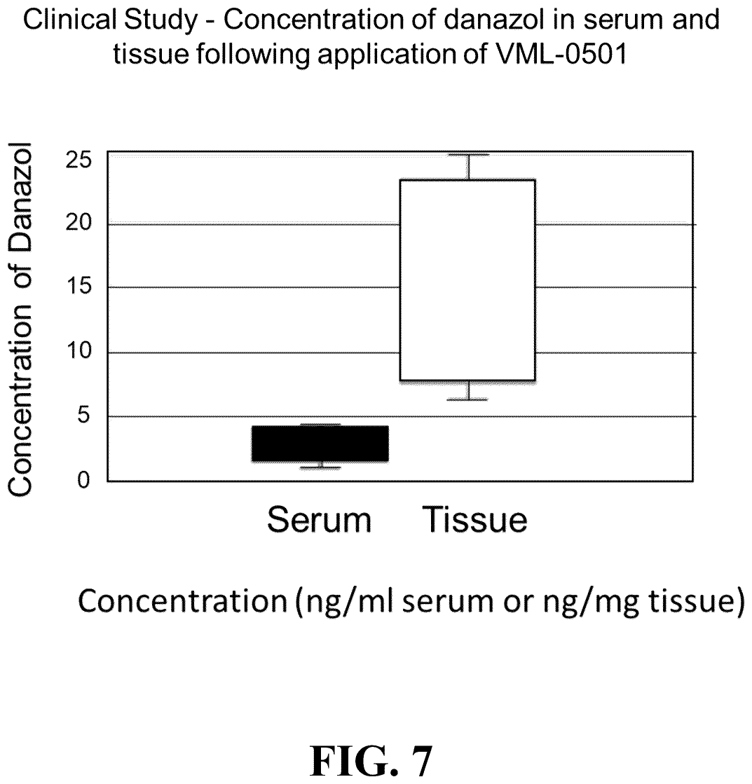

To determine the distribution of danazol in the pelvic tissue between groups, pelvic biopsies (tissue found outside the uterus including ovaries, endometrioma capsules, ovarian fossa, broad ligament, utero sacral ligament and peritoneum) were evaluated from subjects in each group. Danazol was detected in peritoneal fluids and quantified in most samples (12/16) for both groups. Impressively, the concentration of danazol in the tissue biopsies was significantly higher (n=6, p=0.031) in the VML-0501 group as compared to the Danatrol® group. The tissue distribution as shown in FIG. 4 and FIG. 7 is also a superior result because VML-0501 consistently gave higher, sustained concentrations of danazol in the target tissue whereas Danatrol® gave inconsistent and significantly lower danazol concentrations in the target tissue. Before collecting this data it was expected that vaginal application of VML-0501 would have resulted in faster diffusion out of and lower sustained concentrations in the target tissue.

Upon further investigation of the distribution of danazol between groups, the clinical trial data determined that vaginally administered VML-0501 maintained danazol at high and consistent concentrations within the peritoneal tissue via diffusion of the danazol into the peritoneal fluid and reached the pelvic tissue via transvaginal absorption and the counter-current transfer between uterovaginal lymph vessels, veins, and arteries. Therefore, the mechanism by which our vaginally administered composition reached the target pelvic tissue was significantly different from that of orally administered danazol. In turn, vaginal administration of danazol, keeps danazol within the targeted pelvic region and out of systemic circulation (FIG. 7). Thus, vaginally administered VML-0501 unexpectedly and superiorly produced higher sustained target tissue danazol concentrations.

The results described in the Examples above showed that the methods of vaginally administering to a subject a pharmaceutical composition in the form of an emulsion comprising danazol can reduce side effects associated with orally administered danazol. The results described above also show that the methods provided herein also mitigate side effects typically associated with oral administration of danazol. Finally, the results show that vaginal administration of VML-0501 also provides high and sustained concentrations of danazol in the target tissue.

### Example 10: Methods of Treating Pain and Infertility with VML-0501

When improving fertility in endometriosis patients is the primary objective, medical treatment is not recommended. This is because all current medications used in the treatment of endometriosis are hormonal and therefore block ovulation. By contrast, when targeting pain is the primary objective then, medical treatment is beneficial. Endometriosis is viewed as a long-term disease, the natural history of which is unknown, and may require long-term management depending on the patient's age, symptom profile and desire for fertility.

Infertility associated with endometriosis can be related to a mechanical, pelvic anatomical distortion that potentially impairs oocyte ‘pick-up’ by the fallopian tubes. Biochemically, endometriosis can have a detrimental effect on oocyte quality or on endometrial receptivity. Treatment options for women trying to conceive are either expectant management, surgery or assisted reproductive techniques (ARTs).

Provided herein are methods of treating infertility and pelvic pain using VML-0501. The use of low-dose vaginal danazol is a highly effective in the treatment of the painful symptoms, as it aims to reduce inflammation and to immuno-regulate the pain signaling in the peritoneal fluid.

VML-0501 is also used to reduce the size of endometriotic lesions directly by increasing the apoptosis of the endometrial cells outside the utero (cytopathic effect) and by starving lesions locally reducing the expression of aromatase P450 with theoretically no systemic side.

Danazol regulates the functions of normal human endometrial stromal cell subpopulations by modifying endometrial cytokine networks. This results in a “re-programming” effect where everything starts at the eutopic endometrium level.

VML-0501, the polybioadhesive gel provided herein, offers persistence of benefits consistent with direct tissue effects for lower pelvic inflammation. Low serum levels do not affect the hypothalamic-pituitary-axis and protect ovarian functions, minimize systemic effects, and improve fecundability.

Assays can be conducted to determine the most suitable dose of VML-0501 whilst evaluating the safety and efficacy of the dose comparisons. The molecular mechanism of action behind the fertility aspect of Danazol can be explored in further detail with a focus on telomerase activity. Telomerase is active during embryogenesis and is released by cells in the inner lining of the womb during the latter stages of the menstrual cycle in women who are affected by endometriosis, leading to abnormally long telomere length is in these women. Without being bound by a particular theory, administration of vaginal Danazol is expected to have a direct effect on the aberrant telomere homeostasis associated with endometriosis, at different levels (ovarian and eutopic endometrium) and its implication of the telomere pathway in ovarian premature and regular aging. Using VML-0501, the therapy with danazol can improve the reproductive health of thousands of women suffering from endometriosis. It is endeavored that clinical development can establish a safe and efficacious dose for VML-0501 which can maintain ovarian function while treating signs and symptoms of endometriosis.

While embodiments have been shown and described herein, it will be apparent to those skilled in the art that such embodiments are provided by way of example only. Numerous variations, changes, and substitutions can occur to those skilled in the art.

What is claimed is:

1. A method for treating an endometrial disease or condition in a subject, the method comprising:
vaginally administering to the subject a polybioadhesive gel comprising:
(a) danazol or a salt thereof;
(b) one or more water-insoluble bioadhesives;
(c) at least one oleogel; and
(d) at least one aqueous gel,
wherein:
the vaginally administering the polybioadhesive gel reduces systemic concentration of the danazol or the salt thereof in the subject by at least 30-fold relative to oral administration of the danazol or the salt thereof to a subject; and
the vaginally administering the polybioadhesive gel localizes the danazol to a peritoneal tissue of the subject, thereby treating the endometrial disease or condition in the subject.

2. The method of claim 1, wherein the vaginally administering is performed once every 24 hours.

3. The method of claim 1, wherein the danazol or the salt thereof is micronized.

4. The method of claim 1, wherein the subject has or is suspected of having infertility.

5. The method of claim 1, wherein the subject has or is suspected of having endometriosis.

6. The method of claim 1, wherein the vaginally administering reduces a number of endometrial lesions in the subject by at least 10% relative to a number of endometrial lesions in the subject before vaginally administering the polybioadhesive gel.

7. The method of claim 1, wherein the vaginally administering reduces a size of one or more endometrial lesions in the subject by at least 10% relative to a size of one or more endometrial lesions prior to vaginally administering the polybioadhesive gel.

8. The method of claim 1, wherein the polybioadhesive gel is formulated as a liquid emulsion.

9. The method of claim 1, wherein the endometrial disease or condition is endometriosis, adenomyosis, or a combination thereof.

10. The method of claim 1, wherein the polybioadhesive gel does not disrupt ovarian function in the subject.

11. The method of claim 1, wherein the vaginally administering the polybioadhesive gel increases a level of the danazol or the salt thereof in the peritoneal tissue of the subject relative to oral administration of the danazol or the salt thereof to a subject.

12. The method of claim 1, wherein upon vaginal administration, the danazol or the salt thereof diffuses into a peritoneal fluid of the subject, reaching further pelvic tissue via transvaginal absorption, thereby localizing the danazol or the salt thereof to the peritoneal tissue.

13. The method of claim 1, wherein the subject is administered the polybioadhesive gel daily for 5 to 7 consecutive days.

14. The method of claim 1, wherein the danazol or the salt thereof is administered at a dose of 100 mg.

15. The method of claim 1, wherein the danazol or the salt thereof is administered at a dose of 200 mg three times daily for 5 to 7 consecutive days.

16. A method of maintaining ovarian function and reducing pain associated with endometriosis in a subject, the method comprising:
vaginally administering to the subject a polybioadhesive gel comprising:
(a) an active agent or a salt thereof;
(b) one or more water-insoluble bioadhesives;
(c) at least one oleogel; and
(d) at least one aqueous gel, thereby maintaining ovarian function and reducing pain associated with endometriosis in the subject,
wherein:
the vaginally administering the polybioadhesive gel reduces systemic concentration of the active agent or the salt thereof in the subject by at least 30-fold relative to oral administration of the active agent or the salt thereof to a subject;
the active agent or the salt thereof diffuses into a peritoneal fluid of the subject, reaching further pelvic tissue via transvaginal absorption, thereby localizing the active agent to the peritoneal tissue.
